(19) United States
(12) Patent Application Publication
(10) Pub. No.: US 2020/0199533 A1
(43) Pub. Date: Jun. 25, 2020

(54) COMPOSITIONS AND METHODS FOR PREPARING CD34NEG STEM CELLS FOR TRANSPLANT

(71) Applicant: King Abdullah University of Science and Technology, Thuwal (SA)

(72) Inventors: Jasmeen Merzaban, Thuwal (SA); Dina AbuSamra, Thuwal (SA); Asma Al-Amoodi, Thuwal (SA)

(21) Appl. No.: 16/496,880

(22) PCT Filed: Mar. 2, 2018
(86) PCT No.: PCT/IB2018/051357

§ 371 Date: Sep. 23, 2019

Related U.S. Application Data
(60) Provisional application No. 62/475,564, filed on Mar. 23, 2017.

(51) Int. Cl. C12N 5/0789; A61K 35/28

(52) U.S. Cl. CPC C12N 5/0647; C12N 2501/724; A61K 35/28

(57) ABSTRACT

It has been discovered that CD34^{neg} cells, for example HSPCs, can be modified to increase their ability to migrate and to engraft in bone marrow. One embodiment provides a method for modifying CD34^{neg} cells by using glycosyltransferase-programmed stereosubstitution (GPS) to create relevant selectin-binding glycan determinants on the cell surface. For example, the CD34^{neg} cells can be treated with a fucosyltransferase, such as an α(1,3)-linkage-specific fucosyltransferase. Representative enzymes that can be used include, but are not limited to fucosyltransferase VI (FTVI or FucT-6) or fucosyltransferase VII (FTVII of FucT-7). These enzymes specifically places a fucose onto a terminal type 2-lactosamine unit; if that lactosamine is capped with an α(2,3)-linked sialic acid, sLe^{x} is created.

### CROSS REFERENCE TO RELATED APPLICATION

This application claims benefit of and priority to U.S. Provisional Patent Application No. 62/475,564 filed on Mar. 23, 2017, and which is incorporated by reference in its entirety.

### TECHNICAL FIELD OF THE INVENTION

The invention is generally directed to stem cells and methods of using them.

### BACKGROUND OF THE INVENTION

Hematopoietic stem cells (HSCs) are rare cells that are maintained at consistent levels throughout life (self-renewing). They produce hematopoietic progenitor cells (HPCs) that differentiate into every type of mature blood cell within a well-defined hierarchy. Among the different markers for HSCs and HPCs (HSPCs), the cell surface CD34 marker is notoriously known for its unique expression on HSPCs. Clinically the CD34 marker is used to help enrich donor bone marrow with HSPCs prior to bone marrow transplantation, Berenson, R., R. Andrews, W. Bensinger, D. Kalamasz, G. Knitter, C. Buckner, and I. Bernstein, 1988. Antigen CD34+ marrow cells engraft lethally irradiated baboons. Journal of Clinical Investigation. 81:951. However, the role of CD34 as a marker of hematopoietic stem cells is complicated. Many studies suggest a population of dormant human HSCs that are negative for the CD34 marker could acquire the expression of this marker on its progenitors prior to cell division; Bhatia, M., D. Bonnet, B. Murdoch, O. I. Gan, and J. E. Dick. 1998. A newly discovered class of human hematopoietic cells with SCID-repopulating activity. Nat Med. 4:1038-1045; Dooley, D. C., B. K. Oppenlander, and M. Xiao. 2004. Analysis of primitive CD34- and CD34+ hematopoietic cells from adults: gain and loss of CD34 antigen by undifferentiated cells are closely linked to proliferative status in culture. Stem cells. 22:556-569; Goodell, M. A., M. Rosenzweig, H. Kim, D. F. Marks, M. DeMaria, G. Paradis, S. A. Grupp, C. A. Sieff, R. C. Mulligan, and R. P. Johnson. 1997. Dye efflux studies suggest that hematopoietic stem cells expressing low or undetectable levels of CD34 antigen exist in multiple species. Nature medicine. 3:1337-1345; Gotze, K. S., M. Schiemann, S. Marz, V. R. Jacobs, G. Debus, C. Peschel, and R. A. Oostendorp. 2007. CD133-enriched CD34(−) (CD33/CD38/CD71)(−) cord blood cells acquire CD34 prior to cell division and hematopoietic activity is exclusively associated with CD34 expression. Experimental hematology. 35:1408-1414; Osawa, M., K. Hanada, H. Hamada, and H. Nakauchi. 1996. Long-term lymphohematopoietic reconstitution by a single CD34-low/negative hematopoietic stem cell. Science. 273:242-245; and Sonoda, Y. 2008. Immunophenotype and functional characteristics of human primitive CD34-negative hematopoietic stem cells: the significance of the intra-bone marrow injection. J Autoimmun. 30:136-144. In support, studies of gene expression comparing lineage negative fractions of human peripheral blood HSPCs that either express the CD34 antigen or not, imply that CD34 (CD34+ HSPCs) expression is related to cell cycle entry, metabolic activation, and HSPC mobilization and homing whereas the CD34-HSPC subsets are more kinetically and functionally dormant, Manfredini, R., R. Zini, S. Salati, M. Siena, E. Tenedini, E. Tagliafico, M. Montanari, T. Zanocco-Marani, C. Gemelli, T. Vignudelli, A. Grande, M. Fogli, L. Rossi, M. E. Fagioli, L. Catani, R. M. Lemoli, and S. Ferrari. 2005. The kinetic status of hematopoietic stem cell subpopulations underlies a differential expression of genes involved in self-renewal, commitment, and engraftment. Stem cells. 23:496-506. In addition through the enrichment of this CD34-population, Bhatia, M., D. Bonnet, B. Murdoch, O. I. Gan, and J. E. Dick. 1998. A newly discovered class of human hematopoietic cells with SCID-repopulating activity. Nat Med. 4:1038-1045, studies have shown that their homing to and engraftment in the bone marrow is extremely poor compared to their CD34+ counterparts, Nakamura, Y., K. Ando, J. Chargui, H. Kawada, T. Sato, T. Tsuji, T. Hotta, and S. Kato. 1999. Ex vivo generation of CD34(+) cells from CD34(−) hematopoietic cells. Blood. 94:4053-4059; Sonoda, Y. 2008. Immunophenotype and functional characteristics of human primitive CD34-negative hematopoietic stem cells: the significance of the intra-bone marrow injection. J Autoimmun. 30:136-144; and Wang, J., T. Kimura, R. Asada, S. Harada, S. Yokota, Y. Kawamoto, Y. Fujimura, T. Tsuji, S. Ikehara, and Y. Sonoda. 2003. SCID-repopulating cell activity of human cord blood-derived CD34-cells assured by intra-bone marrow injection. Blood. 101:2924-2931. Thus, this potentially very valuable long-term HSC is being ignored in clinical bone marrow transplants worldwide and considered as medical waste while it could potentially be of tremendous benefit.

Quiescent (CD34-) HSCs and neonatal CD34+ umbilical cord blood (UCB) are defective in their ability to migrate to the bone marrow. Even though these cells are currently being ignored in clinical bone marrow transplants worldwide and considered as medical waste, they are believed to have higher multipotency potential compared to the adult HSPCs (CD34+ subsets) from the bone marrow exhibiting very valuable long-term HSCs and even limited numbers of engrafted cells is sufficient for bone marrow reconstitution.

Therefore, it is an object of the invention to provide methods and compositions for improving stem cell migration.

It is another object of the invention to provide biomarkers to identify cancer stem cells.

It is still another embodiment to improve engraftment of bone marrow cells in a subject.

### SUMMARY OF THE INVENTION

It has been discovered that CD34^{neg} cells, for example HSPCs, can be modified to increase their ability to migrate and to engraft in bone marrow. One embodiment provides a method for modifying CD34^{neg} cells by using glycosyltransferase-programmed stereosubstitution (GPS) to create relevant selectin-binding glycan determinants on the cell surface. For example, the CD34^{neg} cells can be treated with a fucosyltransferase, such as an α(1,3)-linkage-specific fucosyltransferase. Representative enzymes that can be used include, but are not limited to fucosyltransferase VI (FTVI or FucT-6) or fucosyltransferase VII (FTVII of FucT-7). These enzymes specifically place a fucose onto a terminal type 2-lactosamine unit; if that lactosamine is capped with an α(2,3)-linked sialic acid, sLe^{x} is created.

One embodiment provides a CD34^{neg} stem cell containing glycoproteins, glycolipids, or a combination thereof, modified to have one or more sLe^{x} structures. Preferably, the stem cell is a hematopoietic stem cell.

Another embodiment provides a CD34^{neg} hematopoietic progenitor cell containing glycoproteins, glycolipids, or a combination thereof, modified to have sLe^{x} structures.

In another embodiment, the CD34^{neg} cell is treated with an α-(1,3)-fucosyltransferase to form the sLe^{x} structures. The α-(1,3)-fucosyltransferase can be FTVI or FTVII.

Still another embodiment provides a pharmaceutical composition containing one more CD34^{neg} cells that are modified to contain one or more sLe^{x} structures.

One embodiment provides a method for improving engraftment of CD34^{neg} cells into bone marrow by contacting the CD34^{neg} cells with an effective amount of a fucosyltransferase to form sLe^{x} structures on glycoproteins, glycolipids, or a combination thereof, of the CD34^{neg} cells. In a preferred embodiment, the CD34^{neg} cells are hematopoietic stem cells or hematopoietic precursor cells. The fucosyltransferase can be FT-VI or FT-VII.

Another embodiment provides a method for increasing hematopoietic cell production in a subject in need thereof, by administering CD34^{neg} cells modified to have one or more sLe^{x} structures. Preferably, the CD34^{neg} cells are HSPCs. In one aspect, the subject has undergone chemotherapy or radiation therapy. In another aspect, the subject has cancer. Preferred cancers are hematological cancers such as AML. In still another aspect, the subject has a chronic infection.

One embodiment provides a method for detecting acute myeloid leukemia (AML) cells by assaying a sample of hematopoietic cells to detect the presence or absence of a non-EseIL reactive form of CD34, wherein the presence of a non-EseIL reactive form of CD34 is indicative of the presence of AML cells. In one aspect, the hematopoietic cells do not contain CD34^{neg} cells. In another aspect, the hematopoietic cells are from a bone marrow sample.

### BRIEF DESCRIPTION OF THE DRAWINGS

FIG. 1A is a bar graph of % of expression CD43, CD44, and PSGL-1 in Lin^{neg}CD38^{neg} and CD34^{neg} cells. FIG. 1B is an autoradiograph of a Western blot showing CD34^{neg} HSPCs do not bind E-selectin (top) and express CD44 (bottom). FIG. 1C is a bar graph of % of E-Ig binding of Lin^{neg}CD38^{neg} and CD34^{neg} cells not treated and treated with FucT-6. FIGS. 1D-1F dot blots showing an overview of the gating strategy used to isolate CD34^{neg}CD38^{neg} and CD34^{pos}CD38^{neg} fractions by FACS sorting from lineage-depleted (Lin^{neg}) bone marrow MNCs.

FIG. 2A is an autoradiograph of a Western blot in which CD34 was immunoprecipitated from HSPC lysates of CD34^{pos} cells isolated from normal UCB cells (CD34^{pos}-UCB), normal bone marrow cells (CD34^{pos}-BM), AML bone marrow cells (CD34^{pos}-AML) or KG1a cells (n=3 patient samples). FIG. 2B is an autoradiograph of the reciprocal immunoprecipitation assay where E-Ig chimera was used first for immunoprecipitation prior to Western blot analysis for CD34 QBend-10 mAb (n=3). FIG. 2C is an autoradiograph of Western blots in which multiple rounds of E-Ig immunoprecipitation were performed on both normal and AML sample cell lysates, and following the clearance of E-Ig reactive bands, the residual lysates were immunoprecipitated with CD34. FIG. 2D is an autoradiograph of Western blots as in 2C and subjected to Western blot analysis for anti-sLe^{x} (HECA-452). Only the first elution after E-Ig immunoprecipitation and the CD34 immunoprecipitation are shown. These blots are representative of n=4 separate experiments. FIG. 2E is a fluorescence micrograph KG1a cells were pretreated with E-Ig chimera or left untreated prior to lipid raft staining with CTB-AF-594. Fixed cells were then stained with CD34 (Cy5; red) and AlexaFluor-488 streptavidin (blue) toward biotinylated anti-human-Ig to detect E-Ig. The colocalized volume was analyzed using Imaris Coloc software. Cell surface labeling with an isotype control or a secondary antibody alone served as background controls (data not shown). Results are representative of two independent experiments, seven fields/experiments.

FIG. 3A is line graph of Response Unit versus time (secs) showing raw data of a KG1a lysate injection on CM5 chip flow cells: blank (black line), immobilized CD34-mAb (4H11) (7360 RU; black dashed line), immobilized CD44-mAb (Hermes3) (5200 RU; gray dashed line), and immobilized isotype control (4377 RU; gray line); the mAb immobilization (step 1) is not shown. Arrows mark the start and end of the lysate injection followed by washing with running buffer. The sensorgrams are uncorrected for the bulk refractive index of the buffer and therefore display a large increase in RU during injection of the lysate. Inset, the result of eluting the captured CD34 from the chip to further confirm specificity were subjected to Western blot analysis and stained for either CD34 (left panel) or PSGL-1 (right panel). FIG. 3B is a line graph of Response Unit versus time (secs) showing normalized values of crude lysate injection from isotype control reference cell. Further normalization was applied to even out the difference in the level of mAb-captured CD34 and CD44 by multiplying the entire sensorgram by a normalization factor that was defined based on its relative response units just prior to injection with E-Ig to the RU in the flow cell with the highest accumulation. Following a brief washing step, the Ca²⁺-dependent binding of both ligand to E-Ig were measured by injecting E-Ig (177 nM) in the presence of 5 mM EDTA and then 1 mM CaCl₂. k_{off} is the dissociation rate constant for CD34 and CD44 from their respective mAb, and k_{off-apparent} is the apparent dissociation rate constant for E-Ig, CD34/E-Ig, or CD44/E-Ig from their respective mAb as well as E-Ig from the complexes CD44⋅Hermes-3-mAb or CD34′4H11-mAb (n=3 independent experiments). FIG. 3C is a bar graph of Bound cells/field from immunopreciptiates of CD34 prepared from lysates of CD34^{pos}-BM and KG1a cells. Adherent CHO-E cells were counted by light microscopy using an ocular grid under 20× magnification (seven fields/slide, two slides/experiment; n=2 independent experiments). FIG. 3D is a bar graph of # of rolling cells for on Western blots of CD34, CD44, CD43, and PSGL-1 immunoprecipitates from KG1a cells stained for HECA-452. CHO-E cells were subsequently perfused over immunoprecipitated glycoproteins at 0.25 dyne/cm². After cell perfusion, the number of rolling cells/field in four distinct fields of view were counted (black bars). CHO-E cells incubated with EDTA (gray bars) and CHO-M (white bars) were used as negative. Results shown reflect the average of rolled cells over the HECA-452 blots of n=7 membrane preparations from four fields of view each. Data are mean±SEM (error bars). *P<0.05, **P<0.01, ***P<0.001.

FIG. 4A is a line graph of Response units (RU) versus time (s) showing Binding of different concentrations of E-Ig to E-selLs (CD34, CD44, CD43, and PSGL-1) expressed in KG1a cell lysate at 150 mM NaCl at the following concentrations: 3.9, 7.8, 15.6, 31.3, 62.5, 125, 250, 500, 1000, and 2000 nM E-Ig injected for 240 s that are spaced by a 60 s buffer washing step. 563-mAb (10800 RU) (Ms IgG1 isotype control, 8320 RU) was immobilized to capture CD34. FIG. 4B is the same as FIG. 4A but Hermes-3-mAb (9300 RU) (Ms IgG2a isotype control, 7700 RU) was immobilized to capture CD44. FIG. 4C is similar to FIG. 4A but KPL-1-mAb (11400 RU) (Ms IgG1 isotype control, 8090 RU) was immobilized to capture PSGL-1. FIG. 4D is similar to FIG. 4A but a polyclonal CD43 Ab (15800 RU) (Goat isotype control, 14450 RU) was immobilized to capture CD43. The sensorgrams are corrected for the bulk refractive index and nonspecific interactions using an isotype control (7300 RU). KD was determined by fitting the binding isotherm using a steady-state model and the RUmax values just prior to the start of the buffer injection where steady-state conditions were nearly met (inset). Data is representative of n=3 independent experiments. FIG. 4E is similar to FIG. 4A and shows binding of different concentrations of E-Ig to CD34 (captured from CD34^{pos}-BM lysate at 50 mM NaCl; bone marrow CD34pos cell lysate was used for real-time immunoprecipitation over surface-immobilized CD34 mAb (563, 6952 RU) (data not shown). The sensorgram shows binding of consecutive injections of similar E-Ig dilutions as in FIG. 4A at 15 μmin for 240 s that are spaced by a 60 s buffer washing step.

FIG. 5A is a bar graph showing flow cytometric analysis of E-selL expression and E-Ig and HECA-452 binding of scrambled and CD34 siRNA-nucleofected KG1a cells (CD34-KD). n=4 experiments depicting the geometric mean fluorescent intensity (G.MFI). FIG. 5B is an autoradiograph of Western blots of scrambled and CD34-KD cell lysates for CD34, E-selLs, and sLe^{x} expression and E-Ig binding using (n=4). FIG. 5C is a line graph of # of rolling cells versus shear stress (dyne/cm²) for scrambled or CD34-KD KG1a cells perfused over CHO-E cell monolayers for 1 min at 0.28 dyne/cm² and then detachment assays were employed by increasing the shear stress stepwise every 15 s. The average number of rolling cells in four distinct fields of view for each experiment (n=4) was counted. FIG. 5D is a line graph of Velocity (μm/s) versus shear stress (dyne/cm²).

FIG. 6A is a line graph of Response unites (RU) versus time (s) in which Scrambled or CD34-KD KG1a cells were each perfused over CHO-E cell monolayers for 1 min at 0.28 dyne/cm² and then detachment assays were employed by increasing the shear stress stepwise every 15 s. The average number of rolling cells in four distinct fields of view for each experiment (n=4) was counted. FIG. 6B is an autoradiograph of Western blot analysis of CD34 immunoprecipitates from HSPC lysates (KG1a or CD34^{pos}-UCB) were either treated with neuraminidase (+) or not (−) and blotted with either E-Ig (top panel) or CD34 (QBend-10, lower panel). FIG. 6C is a line graph of Response Units (RU) versus time (s) showing SPR analysis of the PNGaseF treatment was performed as in FIG. 6A, using 4H11-mAb (6500 RU) or its isotype control (5810 RU). k_{off} and k_{off-apparent} were calculated. FIG. 6D is an autoradiograph of Western blot analysis of CD34 immunoprecipitates treated with PNGase F and subjected to Western blotting for E-Ig (top panel) or CD34 (lower panel). FIG. 6E is a line graph of Response Units (RU) versus time (s) showing SPR analyses of OSGE treatment were performed using 4H11-mAb (9000 RU) or its isotype control (6500 RU). FIG. 6F is an autoradiograph of Western blot analysis of treated CD34 immunoprecipitates performed as in FIG. 6B and FIG. 6D. CD34 (Qbend-10) was used as an internal control to confirm N- and O-glycan removal. All results are representative of n=3 independent experiments.

FIG. 7A is an autoradiograph of Western blots of P-Ig immunoprecipitates from KG1a and CD34^{pos}-BM lysates probing for CD34 (QBend-10 and EP373Y mAb) or PSGL-1 using KPL-1-mAb. Note that CD34 immunoprecipitates were free from any PSGL-1 contamination. (n=3 independent experiment). FIG. 7B is an autoradiograph of Western blots of CD34 and PSGL-1 immunoprecipitates probed with P-Ig (n=3). FIG. 7C is an autoradiograph of Western blots of E-Ig immunoprecipitates of E-sels from KG1 lysate that were eluted with 30 mM EDTA. The eluate was then immunoprecipitated with CD34-mAbs (4H11 and 581) prior to Western blot analysis with CD34, E-Ig, and P-Ig (n=2 independent experiments). FIG. 7D is a bar graph of Bound cells/field for adherent CHO-P cells. CD34 immunoprecipitates were prepared from CD34^{pos}-BM and KG1a lysates and spotted on glass slides to test for CHO-P binding using a Stamper-Woodruff assay. n=2 independent experiments. FIG. 7E is a bar graph of # of rolling cells per field showing Adhesion bar graph representing results obtained for the blot-rolling assay using CHO-P cell rolling at 0.25 dyne/cm² (rolling cells/field) over Western blots of immunoprecipitated CD34, CD44, CD43, or PSGL-1 from KG1a cell lysates. EDTA was used as a buffer (gray bars) or CHO-M cells (white bars). The adhesion bar graph is the average of four fields of view/experiment from n=5; independent experiment and data reported as the mean±SEM (error bars). FIG. 7F is an autoradiograph of CD34 immunoprecipitates from KG1a lysates treated with neuraminidase, OSGE, or PNGaseF or no treatment followed by Western blot analysis with P-Ig. Note that CD34 (QBend-10) was used as internal control (data not shown) (n=3). FIG. 7G is an autoradiograph of KG1a cells were incubated in the presence of 150 mM sodium chlorate for 72 h (left panel). Prior to immunoprecipitation of CD34 or CD34 from KG1a lysates, cells were treated with arylsulfatase at 5 U/ml for 3 h (right panel). The resulting proteins were analyzed by Western blot for P-Ig binding. Note that both treatments abrogated PSGL-1 binding to the KPL-1-mAb from KG1a whole lysates (KPL-1 is sensitive to the loss of sulfation on PSGL-1), while the treatment itself did not significantly affect CD34 protein levels as indicated by EP373Y-mAb staining or PSGL-1 expression (data not shown). Blots are representative of n=3 independent experiments. FIGS. 7H and 71 are line graphs of Response units (RU) versus time (s) showing the Ca²⁺ dependency of P-Ig binding to PSGL-1 and expression of CD34 in KG1a cell lysate at 150 mM NaCl by injecting P-Ig (67 nM) in the presence of 10 mM EDTA followed by consecutive injection of different concentrations of P-Ig in the presence of 2 mM CaCl₂. P-Ig was injected for 170 s interrupted by a 60 s washing buffer step. KPL-1-mAb (11000 RU) Ms IgG₁ isotype control, 5000 RU) was immobilized to capture PSGL-1 (FIG. 7H). 563-mAb (6527 RU) (Ms IgG₁ isotype control, 5000 RU) was immobilized to capture CD34 (FIG. 7I) (n=3).

### DETAILED DESCRIPTION OF THE INVENTION

### I. Definitions

The term “HPSCs” refers to hematopoietic stem/progenitor cells, preferably human cells.

The term “HSC” refers to hematopoietic stem cell. HSC are self-renewing and produce HPCs.

The term “HPC” refers to hematopoietic progenitor cell. HPCs can differentiate into every type of mature blood cell within a well-defined hierarchy.

### II. Modified HSPC Cells

One embodiment provides CD34^{neg} cells, for example HSPCs, modified to have one or more sLe^{x} features to promote engraftment of the cells into bone marrow. CD34^{neg} cells can be modified to have one or more sLe^{x} features by using glycosyltransferase-programmed stereosubstitution (GPS) to create relevant selectin-binding glycan determinants on the cell surface. For example, the CD34^{neg} cells can be treated with a fucosyltransferase, such as an α(1,3)-linkage-specific fucosyltransferase. Representative enzymes that can be used include, but are not limited to fucosyltransferase VI (FTVI or FucT-6) or fucosyltransferase VII (FTVII of FucT-7). These enzymes specifically place a fucose onto a terminal type 2-lactosamine unit; if that lactosamine is capped with an α(2,3)-linked sialic acid, sLe^{x} is created. The sLe^{x} features can be on a polysaccharide, glycoprotein, glycolipid, or a combination thereof. The CD34^{neg} cell can be hematopoietic stem cell, a hematopoietic progenitor cell, or a combination thereof.

A. CD34

For almost 30 years, the cell-surface sialomucin CD34 has been used as a marker to identify and enrich HSPCs in preparation for bone marrow transplantation, Berenson R, Andrews R, Bensinger W, et al. Antigen CD34+ marrow cells engraft lethally irradiated baboons. Journal of Clinical Investigation. 1988; 81(3):951. However, recent studies have revealed that the CD34^{neg} fraction of normal human bone marrow is capable of differentiating into CD34^{pos} subsets that possess a more activated phenotype than was expected, Gotze K S, Schiemann M, Marz S, et al. CD133-enriched CD34(−) (CD33/CD38/CD71)(−) cord blood cells acquire CD34 prior to cell division and hematopoietic activity is exclusively associated with CD34 expression. Exp Hematol. 2007; 35(9):1408-1414; Dooley D C, Oppenlander B K, Xiao M. Analysis of primitive CD34- and CD34+ hematopoietic cells from adults: gain and loss of CD34 antigen by undifferentiated cells are closely linked to proliferative status in culture. Stem Cells. 2004; 22(4):556-569; and Nakamura Y, Ando K, Chargui J, et al. Ex vivo generation of CD34(+) cells from CD34(−) hematopoietic cells. Blood. 1999; 94(12):4053-4059. For example, one major difference that surfaced between these subsets is that the CD34^{neg} fraction suffers from a profound impairment in migration after intravenous transplantation compared to the CD34^{pos} fraction, Dooley D C, Oppenlander B K, Xiao M. Analysis of primitive CD34- and CD34+hematopoietic cells from adults: gain and loss of CD34 antigen by undifferentiated cells are closely linked to proliferative status in culture. Stem Cells. 2004; 22(4):556-569; Sato T, Laver J H, Ogawa M. Reversible expression of CD34 by murine hematopoietic stem cells. Blood. 1999; 94(8):2548-2554; Dao M A, Arevalo J, Nolta J A. Reversibility of CD34 expression on human hematopoietic stem cells that retain the capacity for secondary reconstitution. Blood. 2003; 101(1):112-118; Nielsen J S, McNagny K M. Influence of host irradiation on long-term engraftment by CD34-deficient hematopoietic stem cells. Blood. 2007; 110(3):1076-1077; Sonoda Y. Immunophenotype and functional characteristics of human primitive CD34-negative hematopoietic stem cells: the significance of the intra-bone marrow injection. J Autoimmun. 2008; 30(3):136-144; Lemoli R M, Bertolini F, Petrucci M T, et al. Functional and kinetic characterization of granulocyte colony-stimulating factor-primed CD34-human stem cells. Br J Haematol. 2003; 123(4):720-729; Gao Z, Fackler M J, Leung W, et al. Human CD34+ cell preparations contain over 100-fold greater NOD/SCID mouse engrafting capacity than do CD34-cell preparations. Exp Hematol. 2001; 29(7):910-921; and Verfaillie C M, Almeida-Porada G, Wissink S, Zanjani E D. Kinetics of engraftment of CD34(−) and CD34(+) cells from mobilized blood differs from that of CD34(−) and CD34(+) cells from bone marrow. Exp Hematol. 2000; 28(9):1071-1079. The flow cytometric and Western blot data revealed a more pronounced E-selL activity on the CD34^{pos} subset of the Lin^{neg} CD38^{neg} fraction than on the CD34^{neg} subset. In agreement with these results, a previous study illustrated that bone marrow and fetal liver derived CD34^{pos} subsets rolled over immobilized E-selectin with higher efficiency than CD34^{neg}. Greenberg A W, Kerr W G, Hammer D A. Relationship between selectin-mediated rolling of hematopoietic stem and progenitor cells and progression in hematopoietic development. Blood. 2000; 95(2):478-486. Furthermore, microarray data analysis of CD34^{pos} versus CD34^{neg} subsets revealed exclusive expression of PSGL-1 and CD43 in the positive subset, suggesting that these ligands may be responsible for mediating interactions with E-selectin in this subset. Manfredini R, Zini R, Salati S, et al. The kinetic status of hematopoietic stem cell subpopulations underlies a differential expression of genes involved in self-renewal, commitment, and engraftment. Stem Cells. 2005; 23(4):496-506. Note that the lack of the HCELL glycoform found here indicates that the higher expression of CD44 in the CD34^{neg} subset does not confer E-selectin binding activity, similar to that suggested previously. Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286.

Among several E-selL candidates suggested by MS analysis, CD34 surfaced as a novel and attractive ligand. CD34 is a heavily sialyated O-glycosylated type 1 transmembrane glycoprotein that is negatively charged and is speculated to behave in an anti-adhesive manner, much like its relative mucin CD43. Drew E, Merzaban J S, Seo W, Ziltener H J, McNagny K M. CD34 and CD43 inhibit mast cell adhesion and are required for optimal mast cell reconstitution. Immunity. 2005; 22(1):43-57; Ardman B, Sikorski M A, Staunton D E. CD43 interferes with T-lymphocyte adhesion. Proc Natl Acad Sci USA. 1992; 89(11):5001-5005; and Ohnishi H, Sasaki H, Nakamura Y, et al. Regulation of cell shape and adhesion by CD34. Cell Adh Migr. 2013; 7(5):426-433. Using a number of biochemical and functional assays, for the first time, evidence was provide that both vascular selectins (E- and P-selectin) bind CD34, similar to the other well-described E-selLs, CD44 (i.e. HCELL) and PSGL-1. However, unlike CD34, CD43 has been shown to contribute only modestly to the E-selectin interaction. Merzaban J S, Burdick M M, Gadhoum S Z, et al. Analysis of glycoprotein E-selectin ligands on human and mouse marrow cells enriched for hematopoietic stem/progenitor cells. Blood. 2011; 118(7):1774-1783.

B. Ligand Binding Affinities Expressed on Human HSPCs

The Examples document individual E-selL (CD34, CD44 and PSGL-1) binding affinities expressed on human HSPCs in its native form and show that all ligands display similar dissociation binding constants (K_{D}) with slow on- and off-rate kinetics, with the exception of CD43. A blot-rolling assay confirmed that CD43 has a weaker binding K_{D} by 1.8- to 2.0-fold due to the higher rate by which CD43 dissociates from E-Ig. Furthermore, CD34 was found to play a crucial role in slowing down the velocity of rolling cells at shear stresses ≥3 dyne/cm². These data agree with previous studies that show expression of human CD34 in transgenic mouse thymocytes induces specific binding to human bone marrow endothelial cells compared to control thymocytes that do not express CD34. Healy L, May G, Gale K, Grosveld F, Greaves M, Enver T. The stem cell antigen CD34 functions as a regulator of hemopoietic cell adhesion. Proc Natl Acad Sci USA. 1995; 92(26):12240-12244.

C. Role of CD34 in Guiding HSPCs

The data in the Examples also show that CD34 ligation by E-selectin caused KG1a cell aggregation and CD34 clustering toward lipid rafts, as indicated by an enhanced CTB staining pattern and intensity following E-selectin treatment. A number of studies have suggested that prior to activation, CD34 is homogeneously distributed over the entire cell surface of HSPCs. However, following activation of these cells (via fibronectin or ligation using anti-CD34 antibodies), CD34 redistributes to lipid rafts and in some cases is even polarized toward the uropod, suggesting a role in enhanced homotypic adhesion. Wagner W, Saffrich R, Wirkner U, et al. Hematopoietic progenitor cells and cellular microenvironment: behavioral and molecular changes upon interaction. Stem Cells. 2005; 23(8):1180-1191; Hu M C, Chien S L. The cytoplasmic domain of stem cell antigen CD34 is essential for cytoadhesion signaling but not sufficient for proliferation signaling. Blood. 1998; 91(4):1152-1162; Altrock E, Muth C A, Klein G, Spatz J P, Lee-Thedieck C. The significance of integrin ligand nanopatterning on lipid raft clustering in hematopoietic stem cells. Biomaterials. 2012; 33(11):3107-3118; and Giebel B, Corbeil D, Beckmann J, et al. Segregation of lipid raft markers including CD133 in polarized human hematopoietic stem and progenitor cells. Blood. 2004; 104(8):2332-2338. In fact, studies comparing full-length CD34, a truncated form of CD34 (where most of the cytoplasmic domain is removed), and a chimeric molecule where the cytoplasmic domain of CD34 is fused with the extracellular domain of a cytokine receptor that is not believed to have a role in adhesion, implicate the cytoplasmic domain in the mediation of signaling events causing increased adhesion. Gotze K S, Schiemann M, Marz S, et al. CD133-enriched CD34(−) (CD33/CD38/CD71)(−) cord blood cells acquire CD34 prior to cell division and hematopoietic activity is exclusively associated with CD34 expression. Exp Hematol. 2007; 35(9):1408-1414; and Hu M C, Chien S L. The cytoplasmic domain of stem cell antigen CD34 is essential for cytoadhesion signaling but not sufficient for proliferation signaling. Blood. 1998; 91(4):1152-1162. This role of CD34 in homotypic cell adhesion is significantly abrogated through the tyrosine kinase inhibitor and integrin mAb blockers for LFA-1 and ICAM-1, suggesting a concomitant activation of the LFA-1/ICAM-1 pathway. Hu M C, Chien S L. The cytoplasmic domain of stem cell antigen CD34 is essential for cytoadhesion signaling but not sufficient for proliferation signaling. Blood. 1998; 91(4):1152-1162. These data accompanied with the highly negative structure of CD34 suggest that triggering CD34 signaling events via E-selectin (or anti-CD34 antibodies) contribute to the clustering of CD34 into lipid rafts of hematopoietic cells. This clustering could thereby aid in enhancing adhesion, downstream of selectin binding by activating LFA-1/ICAM-1 integrins and/or Porecha N K, English K, Hangoc G, Broxmeyer H E, Christopherson K W, 2nd. Enhanced functional response to CXCL12/SDF-1 through retroviral overexpression of CXCR4 on M07e cells: implications for hematopoietic stem cell transplantation. Stem Cells Dev. 2006; 15(3):325-333, unmasking adhesiveness of integrins to one another or toward the endothelium. Nielsen J S, McNagny K M. Influence of host irradiation on long-term engraftment by CD34-deficient hematopoietic stem cells. Blood. 2007; 110(3):1076-1077; Drew E, Merzaban J S, Seo W, Ziltener H J, McNagny K M. CD34 and CD43 inhibit mast cell adhesion and are required for optimal mast cell reconstitution. Immunity. 2005; 22(1):43-57; Hu M C, Chien S L. The cytoplasmic domain of stem cell antigen CD34 is essential for cytoadhesion signaling but not sufficient for proliferation signaling. Blood. 1998; 91(4):1152-1162; Majdic O, Stockl J, Pickl W F, et al. Signaling and induction of enhanced cytoadhesiveness via the hematopoietic progenitor cell surface molecule CD34. Blood. 1994; 83(5):1226-1234; and Nielsen J S, McNagny K M. CD34 is a key regulator of hematopoietic stem cell trafficking to bone marrow and mast cell progenitor trafficking in the periphery. Microcirculation. 2009; 16(6):487-496. Here, it was observed that a significant increase in the homotypic aggregation of cells when CD34 was silenced in KG1a cells or when followed by the incubation of the cells with E-Ig or with anti-CD34 mAbs. These results strongly suggest that CD34 has a multifaceted role in guiding HSPCs to E-selectin expressing cells.

D. Selectins

Both vascular selectins are required for human CD34^{pos} cell rolling and homing on bone marrow microvessels, whereby defective rolling is only observed in E-/P-selectin double knockout non-obese/severe combined immunodeficiency mice. Hidalgo A, Weiss L A, Frenette P S. Functional selectin ligands mediating human CD34(+) cell interactions with bone marrow endothelium are enhanced postnatally. J Clin Invest. 2002; 110(4):559-569. Indeed, P-selectin was found to significantly purify committed human HSPCs (CD34^{pos}CD38^{neg} cells) from total bone marrow MNCs. Zannettino A C, Berndt M C, Butcher C, Butcher E C, Vadas M A, Simmons P J. Primitive human hematopoietic progenitors adhere to P-selectin (CD62P). Blood. 1995; 85(12):3466-3477; Wojciechowski J C, Narasipura S D, Charles N, et al. Capture and enrichment of CD34-positive haematopoietic stem and progenitor cells from blood circulation using P-selectin in an implantable device. Br J Haematol. 2008; 140(6):673-681; and Narasipura S D, Wojciechowski J C, Charles N, Liesveld J L, King M R. P-Selectin coated microtube for enrichment of CD34+ hematopoietic stem and progenitor cells from human bone marrow. Clin Chem. 2008; 54(1):77-85. The data herein show that CD34 on HSPCs can function as an alternative P-selectin ligand. Analysis of the glycan requirements of CD34 binding to P-selectin underscore similar characteristic modifications of PSGL-1 binding to P-selectin with one key difference: Wilkins P P, Moore K L, McEver R P, Cummings R D. Tyrosine sulfation of P-selectin glycoprotein ligand-1 is required for high affinity binding to P-selectin. J Biol Chem. 1995; 270(39):22677-22680; and Sako D, Comess K M, Barone K M, Camphausen R T, Cumming D A, Shaw G D. A sulfated peptide segment at the amino terminus of PSGL-1 is critical for P-selectin binding. Cell. 1995; 83(2):323-331, both CD34 and PSGL-1 depend on O-glycosylation and tyrosine sulfation but only PSGL-1 requires sialylation to mediate binding. Moore K L, Stults N L, Diaz S, et al. Identification of a specific glycoprotein ligand for P-selectin (CD62) on myeloid cells. J Cell Biol. 1992; 118(2):445-456. However, the specific glycosylation profile needed for P-selectin recognition remains unknown. For example, P-selectin may bind to sialylated and nonsialylated forms of Le^{x/a} structures. Nelson R M, Dolich S, Aruffo A, Cecconi O, Bevilacqua M P. Higher-affinity oligosaccharide ligands for E-selectin. J Clin Invest. 1993; 91(3):1157-1166. Also, binding of TIM-1 (T-cell immunoglobulin and mucin domain 1), a major P-selectin ligand that controls the rolling of activated T-cells, requires al-3 fucosylation and tyrosine sulfation for efficient binding but not sialylation. Angiari S, Donnarumma T, Rossi B, et al. TIM-1 glycoprotein binds the adhesion receptor P-selectin and mediates T cell trafficking during inflammation and autoimmunity. Immunity. 2014; 40(4):542-553. On a similar note, CD24, a sialoglycoprotein highly expressed in neutrophils as well as at early stages of B-cell development, does not display the sLe^{x} epitope but does carry a HNK-1 sulfate-containing epitope and the O-glycans that are required for such binding. Angiari S, Donnarumma T, Rossi B, et al. TIM-1 glycoprotein binds the adhesion receptor P-selectin and mediates T cell trafficking during inflammation and autoimmunity. Immunity. 2014; 40(4):542-553; Pirruccello S J, LeBien T W. The human B cell-associated antigen CD24 is a single chain sialoglycoprotein. J Immunol. 1986; 136(10):3779-3784; Aigner S, Sthoeger Z M, Fogel M, et al. CD24, a mucin-type glycoprotein, is a ligand for P-selectin on human tumor cells. Blood. 1997; 89(9):3385-3395; and Sammar M, Aigner S, Altevogt P. Heat-stable antigen (mouse CD24) in the brain: dual but distinct interaction with P-selectin and L1. Biochim Biophys Acta. 1997; 1337(2):287-294. In addition to the modifications for P-selectin binding discussed above, CD34 also stained positive for the HNK-1 epitope, suggesting that it may also be important in mediating the binding of CD34 to P-selectin (data not shown). The dissociation binding constants measured here for PSGL-1 and CD34 were 372±5 and 621±4 nM, respectively, which are in accordance with previous studies, which found this value at 320±20 nM (with a k_{off}=1.4±0.1 s⁻¹ and kₒₙ=4.4×10⁶M⁻¹ s⁻¹) for monomeric P-selectin binding to PSGL-1 (isolated from human neutrophils); the injection of membrane-derived P-selectin resulted in slow on and off rates. Mehta P, Cummings R D, McEver R P. Affinity and kinetic analysis of P-selectin binding to P-selectin glycoprotein ligand-1. J Biol Chem. 1998; 273(49):32506-32513. The data has a characteristically similar dissociation binding constant with a slow apparent dissociation rate constant (k_{off-apparent}˜10,000-fold increase) and a smaller apparent association rate constant (kₒₙ₋ₐₚₚₐᵣₑₙₜ˜100-fold reduction). It is suspected that this is primarily due to the use of recombinant P-selectin, which is dimeric in nonionic detergents and therefore binds to PSGL-1 with higher avidity and slower on and off rates than monomeric forms of P-selectin.

E-selL activity depends on a specific posttranslational sLe^{x} glycan decoration, which is detected by HECA-452, of the core protein. For example, PSGL-1 requires core 2 O-linked glycans that are sialylated and fucosylated to bind P- and E-selectins whereas tyrosine sulfate residues are not required for E-selectin binding. Martinez M, Joffraud M, Giraud S, et al. Regulation of PSGL-1 interactions with L-selectin, P-selectin, and E-selectin: role of human fucosyltransferase-IV and -VII. J Biol Chem. 2005; 280(7):5378-5390; Goetz D J, Greif D M, Ding H, et al. Isolated P-selectin glycoprotein ligand-1 dynamic adhesion to P- and E-selectin. J Cell Biol. 1997; 137(2):509-519; and Li F, Wilkins P P, Crawley S, Weinstein J, Cummings R D, McEver R P. Post-translational modifications of recombinant P-selectin glycoprotein ligand-1 required for binding to P- and E-selectin. J Biol Chem. 1996; 271(6):3255-3264. The E-selL activity of CD44 (i.e. HCELL) is conferred by the expression of sialylated, fucosylated binding determinants on both N- and O-glycans Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286; and AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230, while critical sialofucosylated modifications are displayed mainly on O-glycans for CD43. Merzaban J S, Burdick M M, Gadhoum S Z, et al. Analysis of glycoprotein E-selectin ligands on human and mouse marrow cells enriched for hematopoietic stem/progenitor cells. Blood. 2011; 118(7):1774-1783. Work outlined here suggests that sialylated O-glycans rather than N-glycans are the key contributors to CD34 binding to E-selectin.

E. HSC Niches

At least two HSC niches have been described in the bone marrow: an endosteal or osteoblastic niche (quiescence) and a perivascular niche (activated niche). Each is anatomically distinct in the types of regulators that control HSPC proliferation. In the endosteal niche, E-selectin is significantly enriched in the endothelium of the vasculature near the interface with the endosteal region, whereas in the perivascular niche, E-selectin tends to concentrate around the central sinusoidal vasculature. A recent study confirmed slower HSC cycling in mice lacking E-selectin (Sele^{-/-}) compared to wild-type mice; this increased HSC quiescence and selfrenewal potential was further induced by an E-selectin antagonist, GMI-1070, in wild-type mice. Winkler I G, Barbier V, Nowlan B, et al. Vascular niche E-selectin regulates hematopoietic stem cell dormancy, self renewal and chemoresistance. Nat Med. 2012; 18(11):1651-1657. Thus expression of E-selectin in the perivascular niche accelerates HSC proliferation. In support of these previous results, CD34 expression correlates with the active proliferative phase of HSPCs while the negative population represents a more quiescent precursor population in the HSPC hierarchy. Dooley D C, Oppenlander B K, Xiao M. Analysis of primitive CD34- and CD34+ hematopoietic cells from adults: gain and loss of CD34 antigen by undifferentiated cells are closely linked to proliferative status in culture. Stem Cells. 2004; 22(4):556-569; and Ando K, Nakamura Y, Chargui J, et al. Extensive generation of human cord blood CD34(+) stem cells from Lin(−)CD34(−) cells in a long-term in vitro system. Exp Hematol. 2000; 28(6):690-699. The data imply that functional E-selLs are found in the more active CD34^{pos} population, suggesting that these cells prefer to reside in the perivasculature niche where they may act as a control switch for the proliferation/differentiation of HSPCs.

### III. Pharmaceutical Compositions

Still another embodiment provides a pharmaceutical composition containing one more CD34^{neg} cells that are modified to contain one or more sLe^{x} structures. The modified cells can be provided in a container in combination with pharmaceutically acceptable excipient, for example sterile, pH buffered saline or cell culture medium. In certain embodiments, the modified CD34^{neg} cells are cryopreserved.

### IV. Biomarker for AML

The data show that differentially decorated forms of CD34 exist and could be used as a unique marker of leukemic stem cells (LSCs). Following the removal of the E-selectin binding form of CD34 isolated from bone marrow CD34^{pos} AML cells and KG1a cells (but not normal CD34^{pos} cells), a CD34 glycoform lacking sLe^{x} expression was identified. This non-EseIL reactive form of CD34, uniquely expressed on AML cells, could be considered a novel marker for this disease. Future studies using this glycoform of the protein as a target for the generation of mAbs may help to identify and target LSCs in the treatment of leukemia where LSCs are thought to be a major cause of disease relapse. This form of CD34 could provide a means to avoid cell death, as has been implicated by a previous study, suggesting that CD34 expression could be correlated with higher levels of anti-apoptotic proteins contributing to increased resistance of CD34 expressing AML cells to treatments over ones that do not. van Stijn A, van der Pol M A, Kok A, et al. Differences between the CD34+ and CD34-blast compartments in apoptosis resistance in acute myeloid leukemia. Haematologica. 2003; 88(5):497-508.

### V. Methods of Use

One embodiment provides a method for improving engraftment of CD34^{neg} cells into bone marrow by contacting the CD34^{neg} cells with an effective amount of a fucosyltransferase to form sLe^{x} structures on glycoproteins, glycolipids, or a combination thereof, of the CD34^{neg} cells. In a preferred embodiment, the CD34^{neg} cells are hematopoietic stem cells or hematopoietic precursor cells. The cells are also preferably autologous. The fucosyltransferase can be FT-VI or FT-VII.

Typically, CD34neg hematopoietic stem cells, hematopoietic progenitor cells or a combination thereof are isolated from a subject, preferably from a human subjectgra. The CD34^{neg} cells are then treated ex vivo with fucosyltransferase to form sLe^{x} structures on the CD34^{neg} cells. The modified cells can optionally be expanded in cell culture and administered back to the subject. The modified CD34neg cells engraft into the bone marrow of the subject and produce hematopoietic cells.

Another embodiment provides a method for increasing hematopoietic cell production in a subject in need thereof, by administering CD34^{neg} cells modified to have one or more sLe^{x} structures. Preferably, the CD34^{neg} cells are HSPCs. In one aspect, the subject has undergone chemotherapy or radiation therapy. In another aspect, the subject has cancer. Preferred cancers are hematological cancers such as AML. In still another aspect, the subject has a chronic infection.

The modified cells can be administered together with other anti-cancer drugs and imunothapies. Chemotherapeutic agents are well known in the art as well and therapeutics including antibodies to PD-1, PD-L1, and HER2.

One embodiment provides a method for detecting acute myeloid leukemia (AML) cells by assaying a sample of hematopoietic cells to detect the presence or absence of a non-EseIL reactive form of CD34, wherein the presence of a non-EseIL reactive form of CD34 is indicative of the presence of AML cells. In one aspect, the hematopoietic cells do not contain CD34neg cells. In another aspect, the hematopoietic cells are from a bone marrow sample.

### EXAMPLES

### Methods

### Mass Spectrometry Analysis of Cognate E-Selectin Ligands Expressed on Human HSPCs:

E-selectin chimera (E-Ig) immunoprecipitated samples, performed as described in the online Supplemental Methods, were separated on 4-20% SDS-PAGE gels and protein bands were visualized by Simply Blue Safe Stain (Invitrogen). These bands were then reduced, alkylated, and digested in the gel with sequencing grade modified trypsin (Promega); the resultant peptides were extracted using buffer A: 5% acetonitrile (VWR), 95% water, and 0.1% formic acid (Sigma-Aldrich). Following extraction, peptides were dried to approximately 1 μl sample volumes using a speed vacuum, fractionated by nanoflow liquid chromatography, and analyzed using a LTQ Orbitrap mass spectrometer (all acquired from Thermo Scientific). Raw data was converted to Mascot generic format files and searched using the online Mascot database.

### Parallel Plate Rolling Assay of CD34 Knockdown Cells:

CD34 siRNA was predesigned by Ambion silencer select (Applied Bioscience). For each nucleofection, 1×10⁷ cells were pretreated with bromelain (500 μg/ml, 20 min at 37° C.), washed with PBS, gently resuspended in SE buffer mix (Amaxa) containing 500 nM of CD34-targeting (CD34-KD) or nontargeting control (scrambled), and then pulsed with the program EO-100 using a 4D-Nucleofector system (Amaxa). CD34 expression in CD34-siRNA and scrambled nucleofected cells were monitored routinely after 48-72 h to be eligible for experimental use. For the cell binding assay, 1×10⁶CD34-KD or scrambled cells were suspended in HBSS with 10 mM HEPES and 2 mM CaCl₂ and then perfused over an 80% confluent CHO-E cell monolayer at 0.3 dyne/cm² for 60 s followed by stepwise increases every 15 s to a maximum of 4.2 dynes/cm², as previously described in Wiese G, Barthel S R, Dimitroff C J. Analysis of physiologic E-selectin-mediated leukocyte rolling on microvascular endothelium. J Vis Exp. 2009 (24).

### Example 1: CD34⁻ Cells Express CD44 but not PSGL-1 or CD43

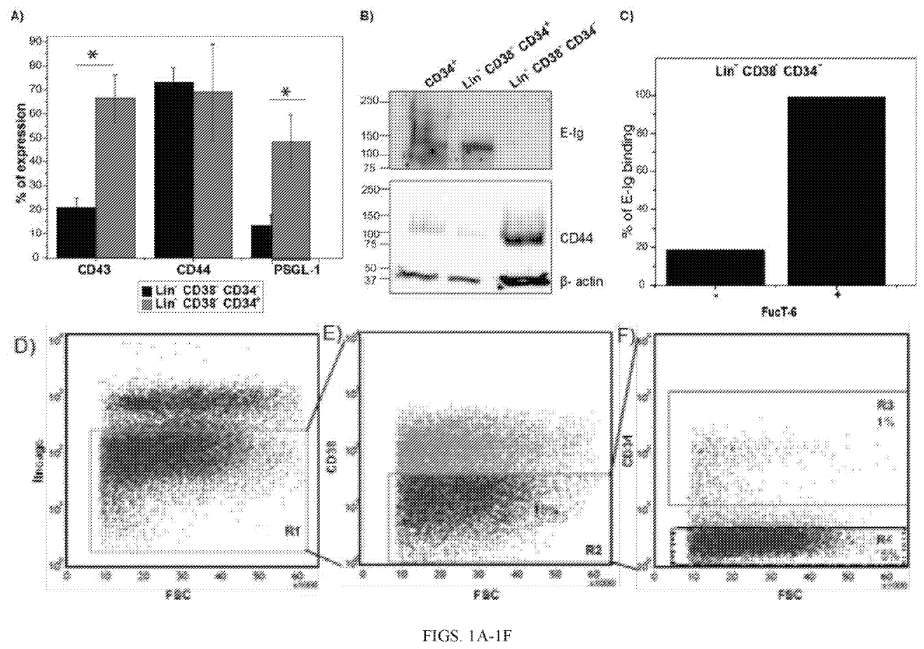

The CD34-fraction of the lineage depleted CD38-bone marrow cells, do express CD44, a cell surface adhesion molecule important for migration, but do not express other molecules important for this process such as PSGL-1 or CD43 (FIG. 1A). Interestingly, studies of human CD34+ bone marrow cells indicate that Step 1 rolling interactions on endothelial cells of the bone marrow (and subsequent extravasation into marrow) are most dependent on the expression of CD44 than other ligands, contrary to HSPCs in mouse, AbuSamra, D. B., A. Al-Kilani, S. M. Hamdan, K. Sakashita, S. Z. Gadhoum, and J. S. Merzaban. 2015. Quantitative Characterization of E-selectin Interaction with Native C D44 and P-selectin Glycoprotein Ligand-1 (PSGL-1) Using a Real Time Immunoprecipitation-based Binding Assay. The Journal of biological chemistry. 290:21213-21230; Dimitroff, C. J., J. Y. Lee, S. Rafii, R. C. Fuhlbrigge, and R. Sackstein. 2001a. CD44 is a major E-selectin ligand on human hematopoietic progenitor cells. The Journal of cell biology. 153:1277-1286; Dimitroff, C. J., J. Y. Lee, K. S. Schor, B. M. Sandmaier, and R. Sackstein. 2001b. differential L-selectin binding activities of human hematopoietic cell L-selectin ligands, HCELL and PSGL-1. The Journal of biological chemistry. 276:47623-47631; and Merzaban, J. S., M. M. Burdick, S. Z. Gadhoum, N. M. Dagia, J. T. Chu, R. C. Fuhlbrigge, and R. Sackstein. 2011. Analysis of glycoprotein E-selectin ligands on human and mouse marrow cells enriched for hematopoietic stem/progenitor cells. Blood. 118:1774-1783. Although CD44 protein was found to be expressed, CD34-HSPCs were not able to bind E-selectin, the counterreceptor expressed on the bone marrow endothelial cells that is responsible for recruiting HSPCs to the bone marrow (FIG. 1B). Glycosyltransferases expressed in the endoplasmic reticulum and Golgi act in an assembly line fashion by adding one glycan at a time to a growing carbohydrate chain decorating a particular glycoprotein. In order for selectin ligands to be formed, glycosyltransferases responsible for creating the sLex structure need to be endogenously expressed and active. As implied by the preliminary data outlined above, CD34-HSPCs do not bind E-selectin likely due to the lack of sLex expression (i.e. HECA-452 negative) suggesting that these glycosyltransferases may not be expressed or active.

Fucosyltransferase-6 (FucT-6), can be used to help create the sLex structure on glycoproteins such as CD44. Ex vivo treatment of stem cells with fucosyltransferases, particularly FucT-6 and FucT-7, increases cell surface sLe^{x} determinants, boosts binding to E-selectin, and enhances homing and engraftment in various mouse models, Merzaban, J. S., J. Imitola, S. C. Starossom, B. Zhu, Y. Wang, J. Lee, A. J. Ali, M. Olah, A. F. Abuelela, S. J. Khoury, and R. Sackstein. 2015. Cell surface glycan engineering of neural stem cells augments neurotropism and improves recovery in a murine model of multiple sclerosis. Glycobiology. 25:1392-1409; Sackstein, R., J. S. Merzaban, D. W. Cain, N. M. Dagia, J. A. Spencer, C. P. Lin, and R. Wohlgemuth. 2008. Ex vivo glycan engineering of CD44 programs human multipotent mesenchymal stromal cell trafficking to bone. Nature medicine. 14:181-187; and Xia, L., J. M. McDaniel, T. Yago, A. Doeden, and R. P. McEver. 2004. Surface fucosylation of human cord blood cells augments binding to P-selectin and E-selectin and enhances engraftment in bone marrow. Blood. 104:3091-3096.

Using recombinant glycosyltransferases on CD34-HSPCs can enhance the migration capacity of these cells through improving their ability to bind E-selectin (FIG. 1C).

FIGS. 1D-1F provide an overview of the gating strategy used to isolate CD34^{neg}CD38^{neg} and CD34^{pos}CD38^{neg} fractions by FACS sorting from lineage-depleted (Lin^{neg}) bone marrow MNCs. The dot blot represents the cell surface expression of a lineage marker cocktail combined by CD7 (FIG. 1D). Cells residing in the negative fraction (R1) were further gated for CD38 negative cells (R2) (FIG. 1E) then subdivided into two subpopulations based on CD34 expression, CD34pos and CD34neg residing in R3 and R4 gates, respectively (FIG. 1F). Data shown is representative of n=4 experiments.

### Example 2: CD34^{pos} HSPCs Express More E-Selectin Ligands than CD34^{neg} HSPCs

Given the requisite expression of E-selectin on bone marrow endothelial cells for HSPC migration and trafficking, Sipkins D A, Wei X, Wu J W, et al. In vivo imaging of specialized bone marrow endothelial microdomains for tumour engraftment. Nature. 2005; 435(7044):969-973, the ability of E-selectin to bind CD34^{pos} versus CD34^{neg} cell populations isolated from HSPCs isolated from human bone marrow (BM-HSPCs) that were depleted of lineage committed cells including those cells expressing CD38 (Lin^{neg} CD38^{neg}) was compared. As expected, more CD34^{neg} than CD34^{pos} cells were found, (15% vs. 1%, respectively), which is in accordance with previous studies, Dooley D C, Oppenlander B K, Xiao M. Analysis of primitive CD34- and CD34+ hematopoietic cells from adults: gain and loss of CD34 antigen by undifferentiated cells are closely linked to proliferative status in culture. Stem Cells. 2004; 22(4):556-569; and Hao Q L, Shah A J, Thiemann F T, Smogorzewska E M, Crooks G M. A functional comparison of CD34+CD38-cells in cord blood and bone marrow. Blood. 1995; 86(10):3745-3753 (FIGS. 1D-1F). Flow cytometric analysis comparing the Lin^{neg} CD38^{neg} CD34^{pos} to Lin^{neg} CD38^{neg} CD34^{neg} fractions showed higher E-selectin binding to the CD34^{pos} population than to the CD34^{neg} population (FIG. 1A). The Western blot evidenced that many more E-selectin ligands (E-selLs) are expressed in the positive population than in the negative population (FIG. 1B). Note that CD34^{pos}-BM cells (isolated from mononuclear cells (MNCs) of human bone marrow using only anti-CD34 microbeads alone; i.e., not Lin^{neg} CD38^{neg}) express more E-selLs than the Lin^{neg} CD38^{neg} CD34^{pos} population (FIG. 1B). The protein concentration of each extract was normalized using a bicinchoninic acid assay and (3-actin staining (FIG. 1B). To determine whether the difference in E-selectin binding between CD34^{pos} and CD34^{neg} was due to differential expression of E-selLs, the expression of the common E-selLs expressed on HSPCs, like CD44, PSGL-1, and CD43, Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286; Katayama Y, Hidalgo A, Furie B C, Vestweber D, Furie B, Frenette P S. PSGL-1 participates in E-selectin-mediated progenitor homing to bone marrow: evidence for cooperation between E-selectin ligands and alpha4 integrin. Blood. 2003; 102(6):2060-2067; and Merzaban J S, Burdick M M, Gadhoum S Z, et al. Analysis of glycoprotein E-selectin ligands on human and mouse marrow cells enriched for hematopoietic stem/progenitor cells. Blood. 2011; 118(7):1774-1783, were analyzed using flow cytometry. As shown in FIG. 1C, CD44 was expressed on both populations. Western blot analysis of the whole-cell lysates further confirmed that CD44 expression in the CD34^{neg} fraction was higher than in the CD34^{pos} population, but did not display HECA-452 reactivity (data not shown) or E-selL activity (FIG. 1B). Thus, the CD44 expressed on the CD34^{neg} fraction is not hematopoietic cell E-/L-selectin ligand (HCELL) while that expressed on the CD34^{pos} fraction did exhibit HCELL characteristics. Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286; and Sackstein R. Fulfilling Koch's postulates in glycoscience: HCELL, GPS and translational glycobiology. Glycobiology. 2016; 26(6):560-570. Other known E-selLs (CD43 and PSGL-1) were expressed at low levels or absent (20±4% vs. 66.5±24% for CD43 and 13±4% vs. 51±11% for PSGL-1 positive cells in CD34^{neg} and CD34^{pos} populations, respectively; p<0.05) (FIG. 1C) Overall, these results support that E-selectin binding is more pronounced on the CD34^{pos} fraction than on the CD34^{neg} fraction in part because of the differential expression of E-selLs between these two populations.

### Example 3: E-Selectin Binds CD34 Expressed on Human HSPCs

To fully elucidate all noncanonical E-selLs expressed on CD34^{pos} HSPCs from the bone marrow (FIG. 1B), a mass spectrometry (MS)-based proteomics approach was used. Among the several hundred ligands recognized by MS with a P value <0.01, the dataset verified the presence of CD44, CD43, and PSGL-1, three well-known HSPC E-selLs (Table 1). Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286; Katayama Y, Hidalgo A, Furie B C, Vestweber D, Furie B, Frenette P S. PSGL-1 participates in E-selectin-mediated progenitor homing to bone marrow: evidence for cooperation between E-selectin ligands and alpha4 integrin. Blood. 2003; 102(6):2060-2067; and Merzaban J S, Burdick M M, Gadhoum S Z, et al. Analysis of glycoprotein E-selectin ligands on human and mouse marrow cells enriched for hematopoietic stem/progenitor cells. Blood. 2011; 118(7):1774-1783.

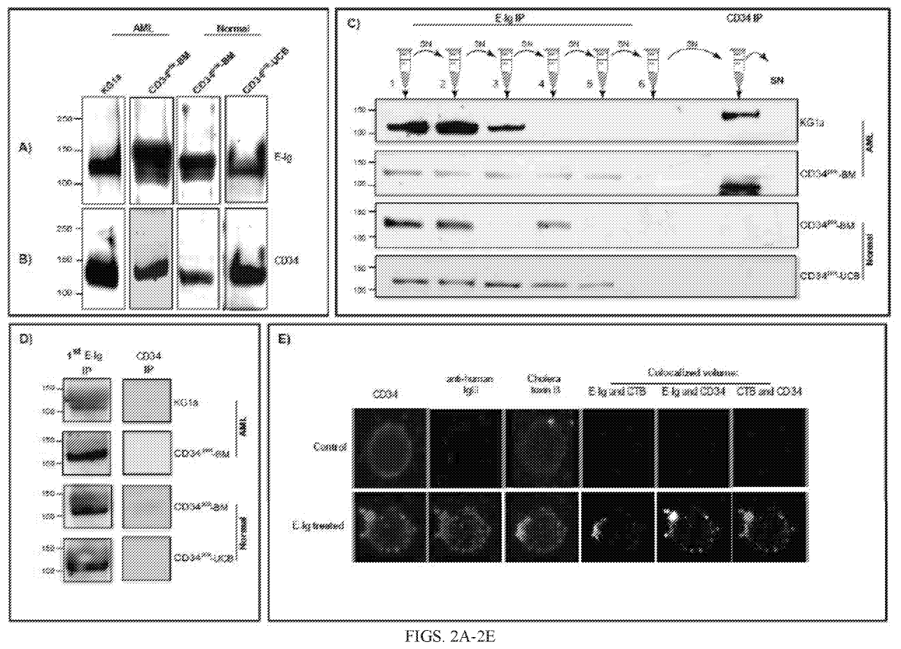

Further, MS data analysis identified the previously unrecognized E-selL, CD34 as a potential ligand on HSPCs. To validate the binding activity of CD34 to E-selectin, CD34 immunoprecipitates were prepared from lysates of CD34^{pos} fractions from normal UCB (CD34^{pos}-UCB and bone marrow (CD34^{pos}-BM) as well as from acute myeloid leukemic (AML) cells (KG1a cell line and bone marrow from AML patient [CD34^{pos}-AML]), all of which had been normalized for protein concentration. Western blots of these immunoprecipitates probed with recombinant E-selectin human immunoglobulin chimeric protein (E-Ig; 1 μg/ml, n=3) revealed a 120 kDa band in all samples tested, confirming that CD34 isolated from HSPCs bound E-selectin (FIG. 2A). Alternatively, results from the reverse experiment verified that CD34 bound E-selectin, suggesting that CD34 isolated from AML cells bound more E-selectin than “equivalent amounts” of CD34 isolated from their normal counterparts (FIG. 2B). Furthermore, an exhaustive immunoprecipitation with E-Ig (six rounds) was performed on each of these cell types. Following the clearance of E-Ig reactive bands, the residual lysate was immunoprecipitated with the anti-CD34 mAb, QBend-10, to confirm that all CD34 expressed on these cells had been removed by immunoprecipitation. Western blot analysis revealed the presence of a CD34 glycoform that was not pulled down by E-Ig on the AML samples (FIG. 2C); this glycoform was not present on normal cells. In addition, this glycoform lacked expression of sialyl Lewis x (sLe^{x}), which is essential for E-selectin binding (FIG. 2D). Finally, confocal microscopy was used to further confirm the interaction of CD34 (CD34-mAb; red) and E-selectin (E-Ig; blue) on KG1a cells that were either pretreated with E-Ig for 1 h or left untreated (FIG. 2E). Using the Imaris Coloc analysis tool, we observed that CD34 appeared to colocalize with E-Ig (FIG. 2E). Interestingly, CD34 appears to colocalize into clusters following a short exposure to E-Ig, suggesting that CD34 may be recruited to lipid rafts following exposure to E-Ig. Indeed, staining with lipid raft markers (CTB, green) indicated that E-Ig induced lipid raft formation and recruited CD34 to lipid rafts after E-Ig binding (FIG. 2E).

TABLE 1
Summary of potential E-selLs expressed on CD34^{pos}-BM cells
identified by MS.

| | | % Coverage | |
|---|---|---|---|
| | | of peptide | |
| Description | Probability | sequence | Peptide Sequence |
| CD34 | 1 | 9.6 | LGILDFTEQDVASHQSYSQK,TSSC[160]AEFKK,LGILDFTEQ |
| | | | DVASHQSYSQK,TSSC[160]AEFK,DRGEGLAR |
| | | | (SEQ ID NO.: 1) |
| Galectin-9B | 0.9997 | 7.3 | FEDGGYVVC[160]NTR SIILSGTVLPSAQR |
| | | | (SEQ ID NO.: 2) |
| Galectin-9 | 0.9998 | 7 | FEDGGYVVC[160]NTR |
| | | | (SEQ ID NO.: 3) |
| CD44/HCELL | 1 | 5.8 | LVINSGNGAVEDR SQEM[147]VHLVNK NLQNVDM[147]K |
| | | | (SEQ ID NO.: 4) |
| Galectin-3 | 1 | 5.6 | ASHEEVEGLVEK LADGGATNQGR |
| | | | AVDTWSWGER (SEQ ID NO. 5) |
| CD43 | 0.9996 | 4.5 | TGALVLSR (SEQ ID NO.: 6) |
| Integrin beta-2 | 1 | 3.9 | TTEGC[160]LNPR YNGQVC[160]GGPGR SSQELEGSC[160]R |
| | | | (SEQ ID NO.: 7) |
| PSGL-1 | 0.9907 | 2.2 | SPGLTPEPR (SEQ ID NO.: 8) |
| FL cytokine | 0.9977 | 1.6 | EMDLGLLSPQAQVEDS (SEQ ID NO.: 9) |
| receptor | | | |
| Integrin alpha-L | 0.9337 | 0.9 | HGGLSPQPSQR |
| | | | (SEQ ID NO.: 10) |

### Example 4: CD34 is an E-selL Under Flow Conditions

To examine whether native CD34 on human HSPCs displays functional E-selL activity under flow conditions, we employed three approaches: the surface plasmon resonance (SPR)-based binding assay developed in our lab, AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230; the Stamper-Woodruff assay, Stamper H B, Jr., Woodruff J J. Lymphocyte homing into lymph nodes: in vitro demonstration of the selective affinity of recirculating lymphocytes for high-endothelial venules. J Exp Med. 1976; 144(3):828-833, and the blot-rolling assay, Fuhlbrigge R C, King S L, Dimitroff C J, Kupper T S, Sackstein R. Direct real-time observation of E- and P-selectin-mediated rolling on cutaneous lymphocyte-associated antigen immobilized on Western blots. J Immunol. 2002; 168(11):5645-5651; and Sackstein R, Fuhlbrigge R. Western blot analysis of adhesive interactions under fluid shear conditions: the blot rolling assay. Methods Mol Biol. 2009; 536:343-354. CD34 binding was compared to the well-established E-selL, CD44 (i.e., HCELL). Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286; and Sackstein R. Fulfilling Koch's postulates in glycoscience: HCELL, GPS and translational glycobiology. Glycobiology. 2016; 26(6):560-570.

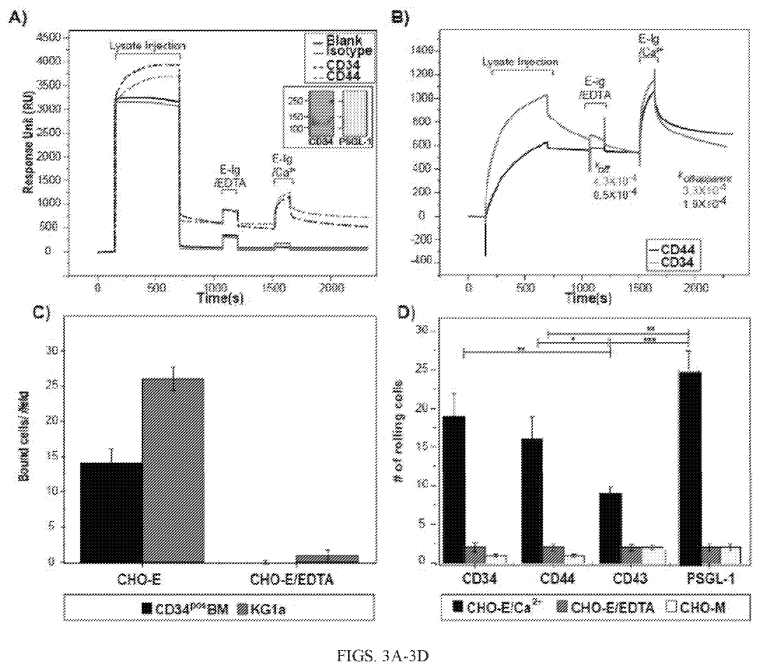

The real-time binding feature of our SPR assay enabled the isolation of endogenously expressed CD34 and CD44 from human HSPC lysate and to measure their direct binding to recombinant E-Ig. Ligand-specific mAbs (4H11 for CD34 and Hermes-3 for CD44) or isotype controls (MsIgG) were first immobilized on a CM-5 sensor chip. An increase in response units (RU) was detected when CD34 and CD44 protein from the HSPC lysate bound to the immobilized antibodies while only residual RU were detected in the control flow cells that were either left blank (no mAb) or were immobilized with isotype control (MsIgG) (FIG. 3A). It should be noted that the CD44 mAb used here fished out a mixture of CD44 glycoforms, AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230, of which only a fraction bound E-selectin. Furthermore, the purity of the captured CD34 was assessed as described previously. AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230. Briefly, a 5 min buffer-washing step following lysate injection was introduced in order to eliminate potential multi-ligand complexes and the and then the bound CD34 was recovered from the surface and by Western blot. Immunostaining with CD34-mAb verified the presence of CD34 (FIG. 3A, inset), while immunostaining for the presence of PSGL-1, a highly expressed E-selL on hematopoietic cells, Katayama Y, Hidalgo A, Furie B C, Vestweber D, Furie B, Frenette P S. PSGL-1 participates in E-selectin-mediated progenitor homing to bone marrow: evidence for cooperation between E-selectin ligands and alpha4 integrin. Blood. 2003; 102(6):2060-2067; and Spertini O, Cordey A S, Monai N, Giuffre L, Schapira M. P-selectin glycoprotein ligand 1 is a ligand for L-selectin on neutrophils, monocytes, and CD34+ hematopoietic progenitor cells. J Cell Biol. 1996; 135(2):523-531, verified the absence of this other potential contaminant (FIG. 3A, inset). As estimated from Equation 1, we observed low ligand-capturing efficiency, likely attributed to the immobilization of the mAb (˜150 kDa; rendering its binding site inaccessible) with only 9.5% and 20.2% for CD34 (120 KDa) and CD44 (˜80 kDa), respectively. The maximum RU reached at the end of the lysate injection (RUₘₐₓ) were 546 for CD34 and 559 RU for CD44 (before normalization; data not shown). An 8.6-fold faster dissociation rate constant (k_{off}) was observed during the washing step for captured CD34 relative to CD44 from the mAb complex (FIG. 3B). Next, the binding of CD34⋅4H11-mAb and CD44⋅Hermes-3-mAb complexes with E-selectin was examined. To assess the specificity of this interaction, E-Ig (177 nM) was injected in the presence of EDTA (5 mM) and confirmed that no interaction occurred in either complex. A marked increase in RU was detected when E-Ig was injected in the presence of Ca²⁺ (FIG. 3B) with no or minimal binding to either isotype or blank controls (FIG. 3A). The apparent dissociation rate constant, which consists of the dissociation of CD34 or CD34⋅E-Ig from 4H11-mAb as well as the dissociation of E-Ig from CD34⋅4H11-mAb, was 3.3×10⁻⁴ s⁻¹ (FIG. 3B).

Immunoprecipitations of CD34 from both CD34^{pos}-BM cells and KG1a cells also supported the presence of adhesive interactions observed with CHO-E cells in Stamper-Woodruff assays displaying 14±2 bound cells to CD34 isolated from CD34^{pos}-BM and 26±2 bound cells to CD34 isolated from KG1a; no binding was observed in the presence of EDTA (FIG. 3C). Moreover, using blot-rolling assays of HECA-452 stained blots of E-selL immunoprecipitates, it was observed that CD34 supported significant rolling of CHO-E cells under physiological shear stress in addition to the well-known E-selLs (CD44, CD43 and PSGL-1) immunoprecipitated from the human KG1a cell lysate (FIG. 3D). CD43 supported the least amount of CHO-E rolling of all the ligands tested. Specificity for E-selectin binding was confirmed by the elimination of binding in the presence of EDTA or by using CHO-mock cells (FIG. 3D). Overall these multiple binding studies revealed that CD34 is a relevant and functional E-selL that may cooperate with other E-selLs in directing human HSPCs to E-selectin expressing sites.

### Example 5: The Equilibrium Binding Constants of E-Selectin to CD44, CD34, and PSGL-1 were Relatively Similar but Varied in their Binding Stoichiometry

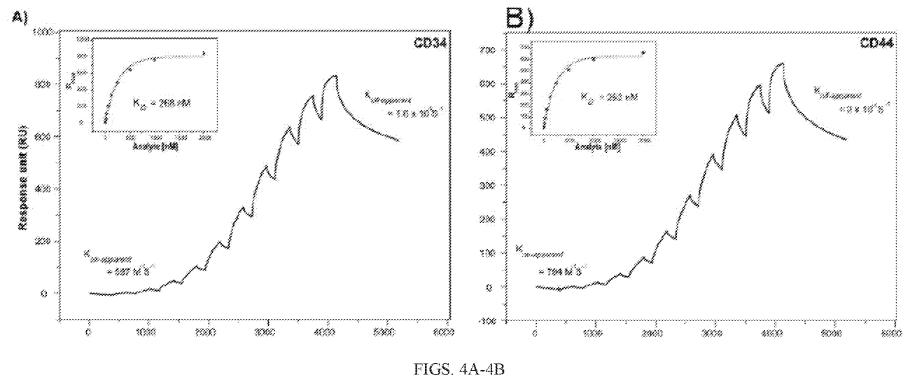

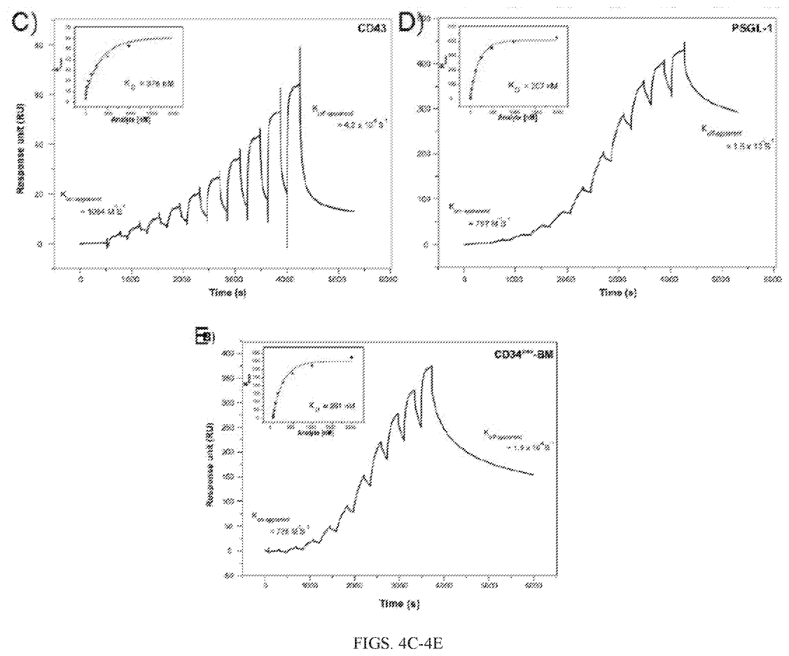

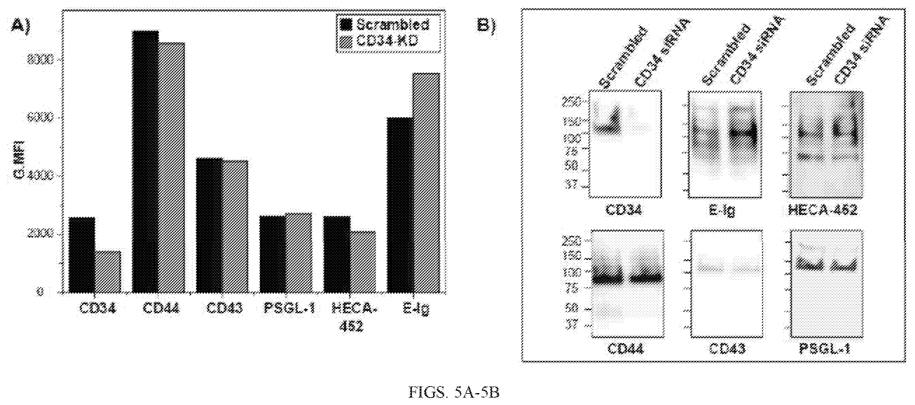

Next, the binding affinities of E-selLs (CD44, CD34, CD43, and PSGL-1) expressed on KG1a cells were directly compared by consecutive E-Ig injection at a physiological NaCl concentration (150 mM) using the SPR-based immunoprecipitation assay. During 5 min of washing at 20 μl/min, captured CD34 and CD43 continuously dissociated from their mAbs such that the amount of protein collected decreased by 20-25% (FIG. 3B). This created an unreliable estimate of the dissociation constant (K_{D}). Therefore, collected CD34′563-mAb and CD43′polyclonal-Ab complexes were covalently linked by inducing another run of amine coupling that included injecting a 1:1 mix of NHS:EDC for 350 s followed by deactivation with ethanolamine for 400 s at 5 μl/min. Using a steady-state model, the dissociation constant (K_{D}), the estimated apparent dissociation rate constant (k_{off-apparent}), and the apparent association rate constant (kₒₙ₋ₐₚₚₐᵣₑₙₜ) were calculated for E-selLs (FIG. 4A and Table. 2). It was observed that CD34, CD44, and PSGL-1 bound to E-Ig similarly with K_{D}=236.7±38, 233±9, and 259±34 nM, respectively, values nearly half that for CD43 (442±47 nM) (Table 2). These results are consistent with the results of the blot-rolling assay, where the numbers of CHO-E cells rolling over CD43 immunoprecipitates were significantly reduced (P<0.05, FIG. 3D).

Assuming 1:1 stoichiometry, E-Ig binding to E-selLs derived from the binding isotherm of the interaction (FIGS. 4A-4D and Table. 2) were 54±17%, 23±0.3%, 19±2%, and 75±3% for CD34, CD44, CD43, and PSGL-1, respectively. These percentages could, however, be upper estimates because we did not rule out the possibility that each ligand could bind more than one E-Ig molecule. Although E-Ig bound E-selLs have similar intrinsic binding behavior (comparable kₒₙ₋ₐₚₚₐᵣₑₙₜ and k_{off-apparent}), PSGL-1 and CD34 were found to express a significantly greater number of E-Ig binding sites than did CD44 or CD43, which could explain the lower rolling behavior of CHO-E over these ligands compared to that rolling over PSGL-1 (P<0.05 and P<0.001, respectively, FIG. 3D). These data suggest that PSGL-1 displayed the highest number of ligand active sites (P<0.001 for CD44 and P<0.01 for CD43) followed by CD34, CD44, and then CD43. Further analysis of the E-selectin binding kinetics of CD34 extracted from primary cells was performed using CD34^{pos}-BM lysate. Due to the relatively low protein concentration in this cell lysate, limited amounts of CD34 (172 RU, data not shown) were pulled out compared to that pulled from the KG1a cell lysate. However, E-Ig binding still displayed a dose-dependent response and using a steady-state model the K_{D} was determined to be 261 nM (FIG. 4E). After the last E-Ig injection, k_{off-apparent}=1.9×10⁻⁴ s⁻¹, which was very similar to that of the CD34 extracted from the KG1a lysate. Thus, kₒₙ₋ₐₚₚₐᵣₑₙₜ≈728 M⁻¹ s⁻¹ was considered a reasonable estimate for CD34. Overall, these results demonstrated relative similarities between the binding affinities of CD34 with cells isolated from either primary CD34^{pos}-BM or KG1a cell lysates, displaying slow on-/slow off-rate kinetics.

TABLE 2
Summary of affinity and kinetic values for E-Ig binding to E-selLs: CD34,
CD44, CD43 and PSGL-1 using a SPR-based assay.

| | CD34 | | | | CD44 | | | |
|---|---|---|---|---|---|---|---|---|
| | 1^{st} | 2^{nd} | 3^{rd} | Mean ± SEM | 1^{st} | 2^{nd} | 3^{rd} | Mean ± SEM |
| K_{D} nM | 145 | 268 | 297 | 236.7 ± 38 | 234 | 252 | 214 | 233 ± 9 |
| K_{off-apparent} S⁻¹ | 2.90E−04 | 1.60E−04 | 3.31E−04 | 2.6E−4 ± 0.4E−4 | 1.10E−04 | 2.00E−04 | 2.73E−04 | 1.9E−4 ± 0.4E−4 |
| Kₒₙ₋ₐₚₚₐᵣₑₙₜ M⁻¹S⁻¹ | 2030 | 597 | 1054 | 1230 ± 346 | 641 | 794 | 1280 | 903 ± 156 |

| | CD43 | | | PSGL-1 | | | |
|---|---|---|---|---|---|---|---|
| | 1^{st} | 2^{nd} | Mean ± SEM | 1^{st} | 2^{nd} | 3^{rd} | Mean ± SEM |
| K_{D} nM | 508.7 | 376 | 442 ± 47 | 342.5 | 207 | 229 | 259 ± 34 |
| K_{off-apparent} S⁻¹ | 4.80E−04 | 4.20E−04 | 4.5E−4 ± 0.2E−5 | 1.50E−04 | 1.65E−04 | 2.62E−04 | 1.9E−4 ± 0.3E−4 |
| Kₒₙ₋ₐₚₚₐᵣₑₙₜ M⁻¹S⁻¹ | 944 | 1063.8 | 1004 ± 43 | 438 | 797 | 1144 | 793 ± 166 |

### Example 6: CD34 Silencing Resulted in Markedly Faster Rolling Velocities at Higher Shear Stress

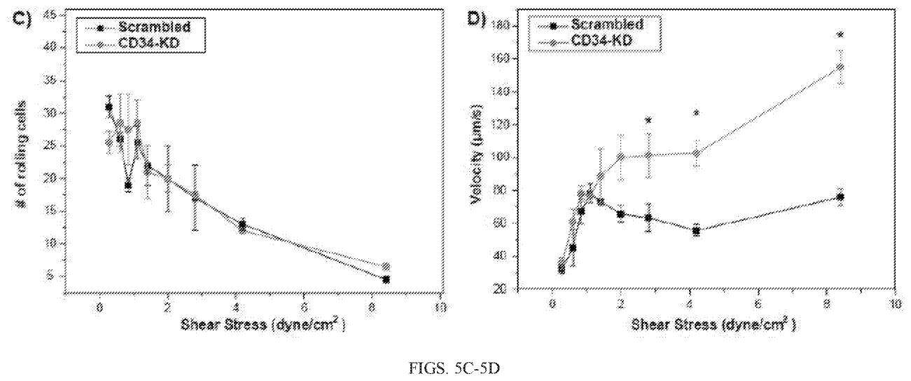

A parallel plate-rolling assay was used to directly measure the relative contribution of CD34 to the overall rolling behavior of HSPCs. Using a knockdown approach, the ability of cells lacking CD34 (CD34-KD) and control cells (scrambled) to tether and roll over E-selectin-expressing cells were compared. Changes in the phenotype and loss of the sLe^{x} epitope that are associated with native HSPCs knockdown cells, Merzaban J S, Burdick M M, Gadhoum S Z, et al. Analysis of glycoprotein E-selectin ligands on human and mouse marrow cells enriched for hematopoietic stem/progenitor cells. Blood. 2011; 118(7):1774-1783, made it favorable to use the HSPC-like cell line KG1a. As measured by flow cytometry, siRNA nucleofection of KG1a cells resulted in a 50% reduction in the surface expression of CD34 relative to scrambled cells (geometric mean fluorescent intensity) (FIG. 5A). Meanwhile, flow cytometry indicated that there were no changes in the expression of other E-selLs or in HECA-452 reactivity of CD34-KD cells compared to scrambled control, but a slight increase in E-Ig staining was observed in CD34-KD cells (FIG. 5A). Western blots of equal amounts of loaded proteins revealed that CD34 expression was substantially reduced, correlating with a slight increase in both HECA-542 and E-Ig staining, especially at ˜100-150 kDa; this observation was not due to an increase in the expression of any of the measured E-selLs (FIG. 5B). It was observed that although the number of rolling cells after perfusing scrambled control or CD34-KD over CHO-E cell monolayers did not vary under any shear stresses tested (FIG. 5C), CD34-KD cells rolled markedly faster than did the control, especially at or above 3 dyne/cm² (postcapillary venule shear; P<0.05; FIG. 5D). Specificity for E-selectin binding was confirmed by treatment of CHO-E cells with a function-blocking mAb (data not shown). These results indicate that CD34 plays a significant role in slowing down the rate at which KG1a cells flow although it does not affect the number of interactions between KG1a cells and CHO-E cells.

### Example 7: CD34 Binding to E-Selectin is Dependent on Sialofucosylated O-Glycans

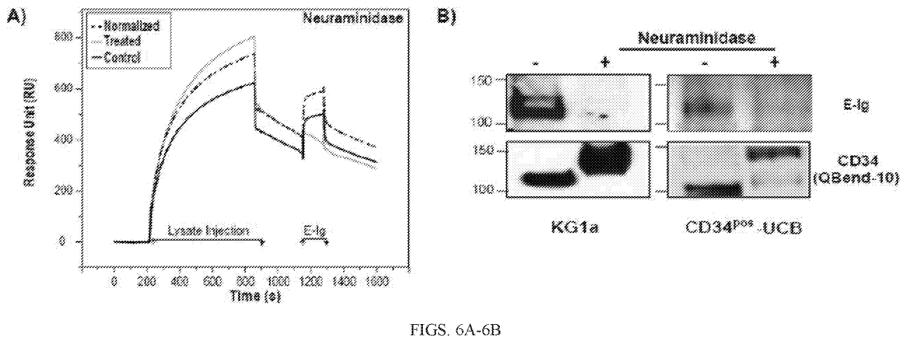

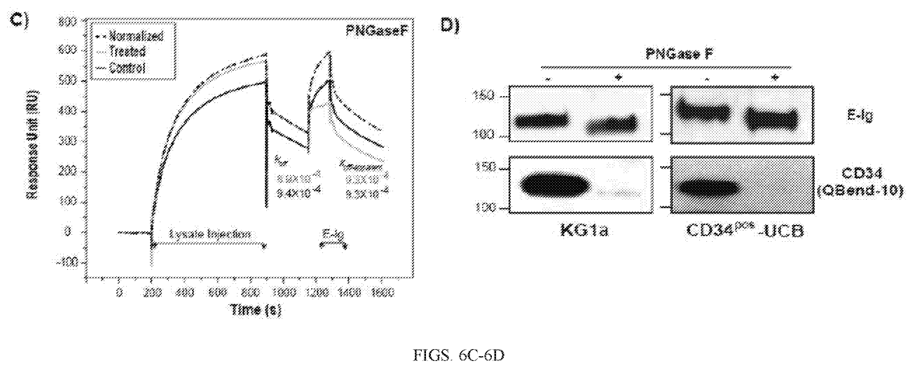

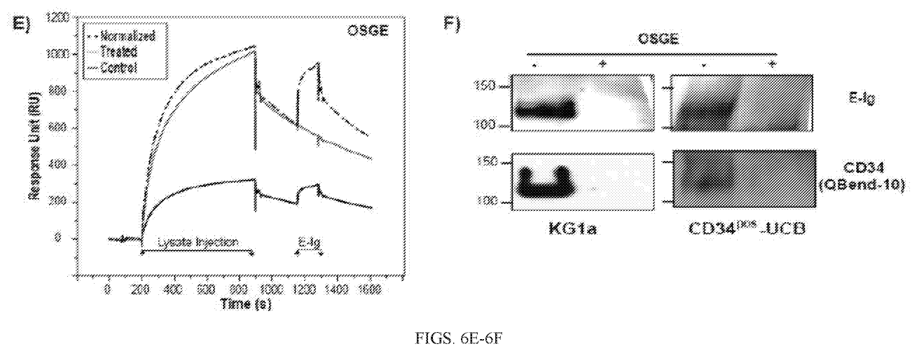

L-selectin is well known to bind sulfated sLe^{x} (6-sulfo-sLe′) capped O- and N-glycans of the CD34 core protein. Hernandez Mir G, Helin J, Skarp K P, et al. Glycoforms of human endothelial CD34 that bind L-selectin carry sulfated sialyl Lewis x capped O- and N-glycans. Blood. 2009; 114(3):733-741. Both a SPR-based immunoprecipitation assay and a Western blot analysis were performed to determine the glycan modifications necessary for CD34 to bind E-selectin following treatment with the glycosidases, OSGE, which removes O-glycans or PNGaseF, which removes N-glycans, or with neuraminidase, which removes sialic acid. To perform a quantitative comparative analysis, we normalized the amount of mAb-captured ligands prior to E-Ig injection. Sensorgrams of E-Ig (354 nM) binding to antibody captured CD34 (CD34⋅4H11-mAb) from either neuraminidase-treated or control lysates showed that binding is eliminated following sialic acid digestion (FIG. 6A). Western blot analysis of treated CD34 immunoprecipitates from KG1a or CD34^{pos}-UCB lysates verified SPR results (FIG. 6B, top panels). The efficiency of neuraminidase to digest sialic acid was evaluated using Western blots and probing with the QBend-10 antibody (Class 2 mAb) to track the shift in the molecular weight of CD34 from ˜120 kDa to ˜150 kDa (FIG. 6B, lower panels). AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230; and Lanza F, Healy L, Sutherland D R. Structural and functional features of the CD34 antigen: an update. J Biol Regul Homeost Agents. 2001; 15(1):1-13. To determine whether the E-selectin binding epitope resides on N- and/or O-glycans, the KG1a lysate was treated with PNGaseF or OSGE. It was found that removing the N-glycans resulted in only a small reduction in RUₘₐₓ (1.4-fold) and a similar dissociation constant (k_{off-apparent}) as the control lysate (FIG. 6C). By contrast, O-glycan removal completely abolished the interaction of E-Ig with the CD34⋅4H11-mAb complex (FIGS. 6E and 6F); results were verified by Western blot analysis (FIGS. 6D and 6F, top panels). The glycoprotease-sensitive CD34-mAb, QBend-10, is sensitive to the removal of O- or N-linked glycans on CD34 and is therefore routinely used to detect the efficiency of glycan removal (FIGS. 6D and F, lower panels). AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230. These findings indicate that, in contrast to CD44 (i.e., HCELL), which displays E-selL determinants on N-glycans and O-glycans, Dimitroff C J, Lee J Y, Rafii S, Fuhlbrigge R C, Sackstein R. CD44 is a major E-selectin ligand on human hematopoietic progenitor cells. J Cell Biol. 2001; 153(6):1277-1286; and AbuSamra D B, Al-Kilani A, Hamdan S M, Sakashita K, Gadhoum S Z, Merzaban J S. Quantitative Characterization of E-selectin Interaction with Native C D44 and P-selectin Glycoprotein Ligand-1 (PSGL-1) Using a Real Time Immunoprecipitation-based Binding Assay. J Biol Chem. 2015; 290(35):21213-21230, CD34 presents sLe^{x} decorations mainly on O-glycans similar to CD43 and PSGL-1. Merzaban J S, Burdick M M, Gadhoum S Z, et al. Analysis of glycoprotein E-selectin ligands on human and mouse marrow cells enriched for hematopoietic stem/progenitor cells. Blood. 2011; 118(7):1774-1783; and Martinez M, Joffraud M, Giraud S, et al. Regulation of PSGL-1 interactions with L-selectin, P-selectin, and E-selectin: role of human fucosyltransferase-IV and -VII. J Biol Chem. 2005; 280(7):5378-5390.

### Example 8: P-Selectin Binds CD34 on Sulfated O-Glycans Expressed on Human HSPCs

E- and P-selectin are expressed on human bone marrow endothelial cells. Lehr J E, Pienta K J. Preferential adhesion of prostate cancer cells to a human bone marrow endothelial cell line. J Natl Cancer Inst. 1998; 90(2):118-123; and Schweitzer K M, Drager A M, van der Valk P, et al. Constitutive expression of E-selectin and vascular cell adhesion molecule-1 on endothelial cells of hematopoietic tissues. Am J Pathol. 1996; 148(1):165-175. To date, the only ligand known to bind all three selectins (E-/P- and L-selectin) is PSGL-1. Katayama Y, Hidalgo A, Furie B C, Vestweber D, Furie B, Frenette P S. PSGL-1 participates in E-selectin-mediated progenitor homing to bone marrow: evidence for cooperation between E-selectin ligands and alpha4 integrin. Blood. 2003; 102(6):2060-2067; Spertini O, Cordey A S, Monai N, Giuffre L, Schapira M. P-selectin glycoprotein ligand 1 is a ligand for L-selectin on neutrophils, monocytes, and CD34+ hematopoietic progenitor cells. J Cell Biol. 1996; 135(2):523-531; and Levesque J P, Zannettino A C, Pudney M, et al. PSGL-1-mediated adhesion of human hematopoietic progenitors to P-selectin results in suppression of hematopoiesis. Immunity. 1999; 11(3):369-378.

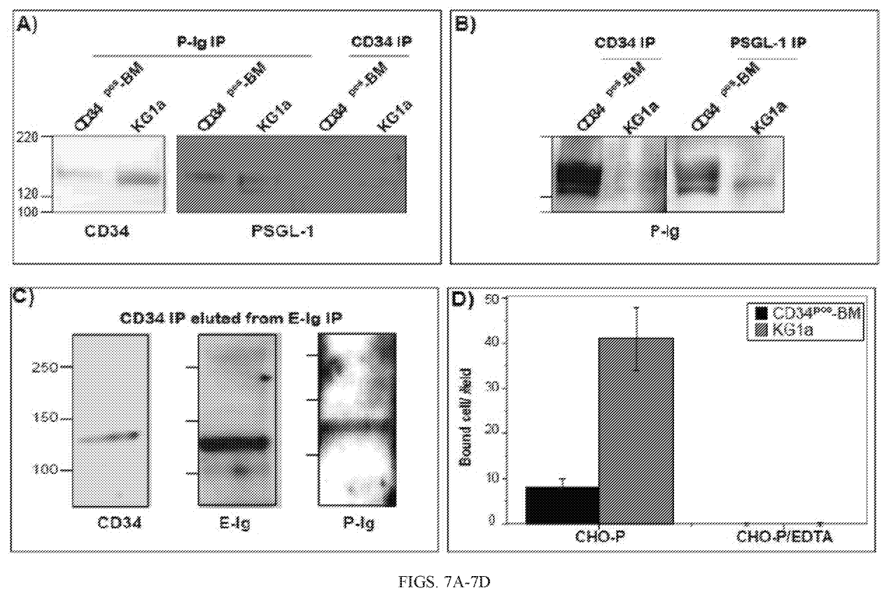

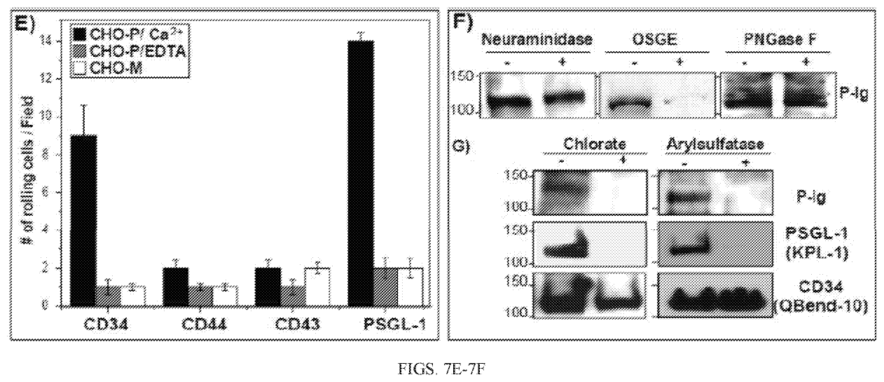

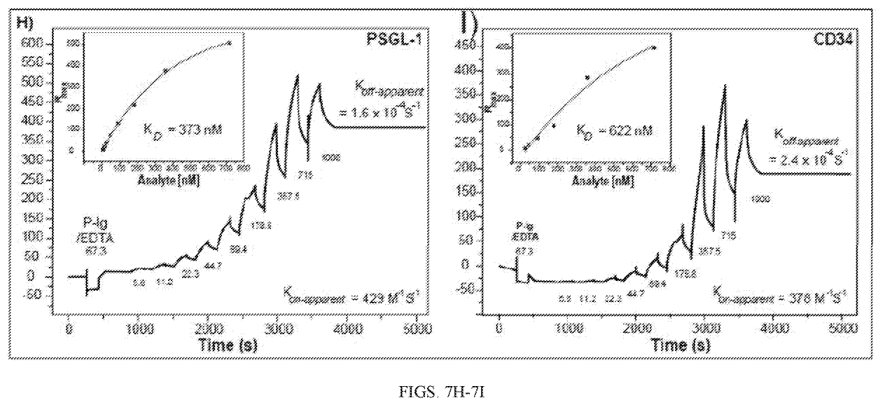

Recombinant P-selectin human immunoglobulin chimeric protein (P-Ig) was used to immunoprecipitate P-selectin ligands from KG1a or CD34^{pos}-BM lysates, and subsequently were blotted for CD34 (EP373Y and QBend-10-mAb) or for PSGL-1 (KPL-1-mAb). CD34 and PSGL-1 were pulled out using P-Ig and no PSGL-1 was found within the CD34 immunoprecipitate (FIG. 7A). In addition, reciprocal studies also confirmed that CD34 and PSGL-1 immunoprecipitated from lysates of KG1a and CD34^{pos}-BM bound P-selectin (FIG. 7B), Intriguingly, two molecular weight forms (between 120-130 kDa) of CD34 appeared to be able to bind P-selectin in the HSPC lysates (FIG. 7B), therefore, to determine whether E-selectin recognizes the same CD34 glycoform as P-selectin, we eluted E-selLs following E-Ig immunoprecipitation of KG1a lysate using EDTA and subsequently immunoprecipitated this elution with CD34-mAb. As shown in FIG. 7C, the higher molecular weight form of CD34 (˜130 kDa) is both an E- and a P-selectin ligand, denoting this molecular weight glycoform of CD34 as a ligand that is able to bind both vascular selectins on HSPCs. Furthermore, CD34 immunoprecipitated from CD34^{pos}-BM and KG1a cells supported CHO-P cell rolling (8±2 cf. 41±7 bound cells per field, respectively) using Stamper-Woodruff assays, whereas no binding was observed with the EDTA control (FIG. 7D). Moreover, among the other E-selLs (CD34, CD44, CD43 and PSGL-1) only CD34 and PSGL-1 exhibited functional binding activity to CHO-P in blot-rolling assays (FIG. 7E). Glycoprotease treatment of CD34 immunoprecipitates from KG1a cells revealed neither treatment with PNGaseF nor with neuraminidase inhibited the binding of P-Ig to CD34, suggesting that N-glycans and sialic acid are not critical for P-selectin binding. Alternatively, OSGE treatment significantly reduced P-Ig staining suggesting that O-glycans are essential for binding. To determine whether sulfation of CD34 is critical for P-selectin binding, we cultured KG1a cells in the presence or absence of chlorate, a metabolic inhibitor of the main sulfate donor on both tyrosine residues and glycoconjugates, Mintz K P, Fisher L W, Grzesik W J, Hascall V C, Midura R J. Chlorate-induced inhibition of tyrosine sulfation on bone sialoprotein synthesized by a rat osteoblast-like cell line (UMR 106-01 BSP). J Biol Chem. 1994; 269(7):4845-4852, prior to the preparation of cell lysates. As shown in FIG. 7G, CD34 immunoprecipitates after chlorate treatment were not able to bind P-selectin compared with the control (left panel). Furthermore, CD34 immunoprecipitates treated with arylsulfatase, an enzyme that releases sulfates from tyrosine residues but not carbohydrates, Snapp K R, Ding H, Atkins K, Warnke R, Luscinskas F W, Kansas G S. A novel P-selectin glycoprotein ligand-1 monoclonal antibody recognizes an epitope within the tyrosine sulfate motif of human PSGL-1 and blocks recognition of both P- and L-selectin. Blood. 1998; 91(1):154-164; and Wilkins P P, Moore K L, McEver R P, Cummings R D. Tyrosine sulfation of P-selectin glycoprotein ligand-1 is required for high affinity binding to P-selectin. J Biol Chem. 1995; 270(39):22677-22680, abrogated P-selectin binding to CD34 compared to the control (treated with buffer alone) (FIG. 7G, right panel). Complete cleavage of sulfated tyrosine residues was confirmed by the loss of anti-PSGL-1-mAb, KPL-1 epitope (FIG. 7G, middle panels), which recognizes the tyrosine-sulfated motif without affecting the overall amount of PSGL-1 protein recognized by PL-2 (data not shown) or the CD34 protein level recognized by QBend-10-mAb following treatment (FIG. 7D, lower panels). Snapp K R, Ding H, Atkins K, Warnke R, Luscinskas F W, Kansas G S. A novel P-selectin glycoprotein ligand-1 monoclonal antibody recognizes an epitope within the tyrosine sulfate motif of human PSGL-1 and blocks recognition of both P- and L-selectin. Blood. 1998; 91(1):154-164. In summary, these data imply that unlike PSGL-1 binding to P-selectin, which requires sialylated glycans, Wilkins P P, Moore K L, McEver R P, Cummings R D. Tyrosine sulfation of P-selectin glycoprotein ligand-1 is required for high affinity binding to P-selectin. J Biol Chem. 1995; 270(39):22677-22680; and Sako D, Comess K M, Barone K M, Camphausen R T, Cumming D A, Shaw G D. A sulfated peptide segment at the amino terminus of PSGL-1 is critical for P-selectin binding. Cell. 1995; 83(2):323-331, the binding of CD34 to P-selectin is not dependent on sialylation but does require O-glycans and tyrosine sulfation.

Finally, the binding kinetics of CD34 in comparison to PSGL-1 were investigated using our SPR-based immunoprecipitation assay. Due to high background binding of P-Ig to the flow cell when these experiments were run under similar conditions to those mentioned above for E-Ig, optimization of the running buffer used on control flow cells was required (see online Supplemental Methods). As shown in FIG. 7H, P-selectin binding to PSGL-1 and CD34 was Ca²⁺ dependent because binding was abolished when injected in the presence of EDTA. The normalized signals produced by a representative series of P-Ig injections at different concentrations over PSGL-1⋅KPL-1-mAb and CD34⋅563-mAb are shown in FIG. 7H, and their specific equilibrium responses were blotted against injected concentrations of P-Ig over PSGL-1 and CD34, respectively (FIG. 7H, inset). Using a steady-state model, the mean dissociation binding constant of P-Ig to PSGL-1 at 25° C. was found to be K_{D}=372±5 nM while that of P-Ig to CD34 was ˜1.7-fold weaker with K_{D}=621±4 nM. This difference is attributed to a difference in the dissociation rates for CD34 (k_{off-apparent}3×10⁻⁴±0.1×10⁻⁴ s⁻¹) and PSGL-1 (k_{off-apparent}2×10⁴±0.3×10⁻⁴ s⁻¹) because the association rates were similar (473.5±32 M⁻¹ s⁻¹ and 451.5±52 M⁻¹ s⁻¹ for PSGL-1 and CD34, respectively).

1. A CD34^{neg} stem or hematopoietic progenitor cell comprising glycoproteins, glycolipids, or a combination thereof, modified to have sLe^{x} structures.

2. The stem cell of claim 1, wherein the stem cell is a hematopoietic stem cell.

3. The cell of claim 1, wherein the cell is a CD34^{neg} hematopoietic progenitor cell comprising glycoproteins, glycolipids, or a combination thereof, wherein the cell is modified to have sLe^{x} structures.

4. The cell of claim 1, wherein the cell is treated with an α-(1,3)-fucosyltransferase to form the sLe^{x} structures.

5. The cell of claim 4, wherein the α-(1,3)-fucosyltransferase is FT-VI or FT-VII.

6. A pharmaceutical composition comprising the CD34^{neg} cell of claim 1.

7. A method for improving engraftment of CD34^{neg} cells comprising:
contacting the CD34^{neg} cells with an effective amount of a glycosyltransferase to form sLe^{x} structures on glycoproteins, glycolipids, or a combination thereof, of the CD34^{neg} cells.

8. The method of claim 5, wherein the stem cells are hematopoietic stem cells or hematopoietic precursor cells.

9. The method of claim 7, wherein the glycosyltransferase is an α-(1,3)-fucosyltransferase.

10. The method of claim 9, wherein the α-(1,3)-fucosyltransferase is FT-VI or FT-VII.

11. A method for increasing hematopoietic cell production in a subject in need thereof, comprising administering the cells of claim 1.

12. The method of claim 11, wherein the subject has undergone chemotherapy or radiation therapy.

13. The method of claim 11, wherein the subject has cancer.

14. The method of claim 13, wherein the cancer is a hematological cancer.

15. The method of claim 11, wherein the subject has a chronic infection.

16. A method for detecting acute myeloid leukemia (AML) cells comprising assaying a sample of hematopoietic cells to detect the presence or absence of a non-EseIL reactive form of CD34, wherein the presence of a non-EseIL reactive form of CD34 is indicative of the presence of AML cells.

17. The method of claim 16 wherein the hematopoietic cells do not contain CD34^{neg} cells.

18. The method of claim 16, wherein the hematopoietic cells are from a bone marrow sample.

19. The cells of claim 1, wherein the cells are autologous.

20. A method for increasing hematopoietic cell production in a subject in need thereof, comprising administering the composition of claim 6.


SEQUENCE LISTING (10 sequences)

SEQ ID NO: 1 - protein, 65 aa, Artificial Sequence
  Synthetic
LGILDFTEQDVASHQSYSQKTSSCAEFKKLGILDFTEQDVASHQSYSQKTSSCAEFKDRGEGLAR

SEQ ID NO: 2 - protein, 26 aa, Artificial Sequence
  Synthetic
FEDGGYVVCNTRSIILSGTVLPSAQR

SEQ ID NO: 3 - protein, 12 aa, Artificial Sequence
  Synthetic
FEDGGYVVCNTR

SEQ ID NO: 4 - protein, 31 aa, Artificial Sequence
  Synthetic
LVINSGNGAVEDRSQEMVHLVNKNLQNVDMK

SEQ ID NO: 5 - protein, 33 aa, Artificial Sequence
  Synthetic
ASHEEVEGLVEKLADGGATNQGRAVDTWSWGER

SEQ ID NO: 6 - protein, 8 aa, Artificial Sequence
  Synthetic
TGALVLSR

SEQ ID NO: 7 - protein, 30 aa, Artificial Sequence
  Synthetic
TTEGCLNPRYNGQVCGGPGRSSQELEGSCR

SEQ ID NO: 8 - protein, 9 aa, Artificial Sequence
  Synthetic
SPGLTPEPR

SEQ ID NO: 9 - protein, 16 aa, Artificial Sequence
  Synthetic
EMDLGLLSPQAQVEDS

SEQ ID NO: 10 - protein, 11 aa, Artificial Sequence
  Synthetic
HGGLSPQPSQR